(19) United States
(12) Patent Application Publication
(10) Pub. No.: US 2018/0353507 A1
(43) Pub. Date: Dec. 13, 2018

(54) METHODS OF PREVENTING OR TREATING HYPOGLYCEMIA BY ADMINISTERING A GPR119 AGONIST

(71) Applicant: Merck Sharp & Dohme Corp., Rahway, NJ (US)

(72) Inventors: David E. Kelley, Westfield, NJ (US); Ping Liu, Westfield, NJ (US); Harold B. Wood, Westfield, NJ (US); Scott D. Edmondson, Clark, NJ (US); Hideo Makimura, Scotch Plains, NJ (US); Xiaoyan Li, Cranford, NJ (US); Timothy Joseph Kowalski, Madison, NJ (US)

(73) Assignee: Merck Sharp & Dohme Corp., Rahway, NJ (US)

(21) Appl. No.: 15/780,462

(22) PCT Filed: Dec. 13, 2016
(86) PCT No.: PCT/US16/66245

§ 371 Date: May 31, 2018

Related U.S. Application Data
(60) Provisional application No. 62/268,212, filed on Dec. 16, 2015.
(60) Provisional application No. 62/344,683, filed on Jun. 2, 2016.

(51) Int. Cl. A61K 31/506; A61K 31/445; A61K 31/454; A61K 31/4545; A61P 1/18

(52) U.S. Cl. CPC A61K 31/506; A61K 31/445; A61P 1/18; A61K 31/4545; A61K 31/454

(57) ABSTRACT

Use of a GPR119 agonist in the prevention or treatment of hypoglycemia is disclosed, addition, pharmaceutical compositions and combinations of a GPR1 19 agonist with insulin, insulin analog, insulin secretagogue or or other drug(s) that reduces plasma glucose levels to below 70 mg/dL described herein.

### BACKGROUND OF THE INVENTION

Diabetes refers to a disease process derived from multiple causative factors which is characterized by elevated levels of plasma glucose (hyperglycemia) in the fasting or postprandial state. Elevated levels of plasma glucose can result in various symptoms, including impacted (blurry) vision, excessive thirst, fatigue, hunger, frequent urination and weight loss. Left untreated, hyperglycemia can lead to serious vision problems, sores and infections in the feet and skin, nerve damage, and cardiovascular complications. Insulin and sulfonylurea drugs, as well as other drugs which raise insulin levels independently of blood glucose, constitute an effective and most commonly used means to counteract hyperglycemia. Unfortunately, one complication associated with the treatment of diabetes with these drugs is the onset of hypoglycemia.

Hypoglycemia is a clinical syndrome characterized by low plasma glucose, symptomatic sympathetic nervous system stimulation and CNS dysfunction. Initially, an individual with progressively declining plasma glucose concentrations may present with any of a series of physiological or behavioral responses selected from sweating, nausea, warmth, anxiety, tremulousness, palpitations, and possibly hunger and paresthesias. These symptoms typically occur starting at a plasma glucose level of about 60 mg/dL (3.3 mmol/L) or below. If not treated, this may progress to headache, blurred or double vision, confusion, difficulty speaking, seizures, and coma as a result of the insufficient glucose supply to the brain. These CNS symptoms will typically occur starting at or below 50 mg/dL (2.8 mmol/L).

Certain individuals who have plasma glucose levels in these ranges do not present with the initial warning signs, i.e., sweating, nausea, etc., and may lapse unknowingly into the severe CNS symptoms. This is because individuals can develop hypoglycemia unawareness as a result of repeated episodes of hypoglycemia. These individuals lose a key component of the body's early response to the low plasma glucose—the epinephrine response—which signals the liver to produce more glucose and triggers the initial warning signs of hypoglycemia.

The danger inflicted by repeated episodes of hypoglycemia is significant, and further aggravated by the fact that the early warning signs are not always associated with hypoglycemia. Given the serious sequelae of hypoglycemia, complex physiological systems have evolved to prevent its development. In individuals without diabetes mellitus, the first defense against hypoglycemia is the reduction in the secretion of endogenous insulin. Subsequently, stimulation of several hormones collectively referred to as “counter-regulatory” hormones occurs. These hormones include glucagon, cathecholamines, cortisol and growth hormone. The counter-regulatory hormones are secreted in a hierarchical manner. Among them, glucagon is considered the primary counter-regulatory hormone given its early rise (within minutes) in response to hypoglycemia and its known effects to stimulate endogenous glucose production via glycogenolysis and gluconeogenesis. Cortisol and growth hormone exert their counter-regulatory effects over the longer term, manifesting their effects on glucose utilization and glucose production over several hours. In patients with diabetes mellitus who are treated with exogenous insulin, insulin analogues, insulin secretagogues, or other compounds that increase insulin release, persistent hyperinsulinemia occurs despite hypoglycemia. Furthermore, patients with type 1 diabetes mellitus (T1DM) manifest a blunted glucagon response specifically to hypoglycemia, making them even more likely to develop and progress into severe hypoglycemia. While T1DM subjects have destruction of insulin producing β-cells in the pancreatic islets, the glucagon producing α-cells persist and in some circumstances experience hypertrophy. These α-cells are able to synthesize and secrete glucagon in response to other physiologic stimuli including high protein meals but do not do so in response to hypoglycemia in T1DM.

In practice, individuals experiencing the early warning signs are typically not in the physician's office where immediate measures can be taken to counteract the progressively declining plasma glucose levels. While the current treatment regimen involves the provision of glucose, there is clearly a need for agents able to effectively and appropriately treat and preferably prevent hypoglycemia in patients being treated with anti-diabetic medications which raise insulin levels independently of blood glucose, such as insulin and sulfonylurea drugs.

While the G-protein coupled receptor GPR119 was originally recognized to be predominantly localized to pancreatic islet β cells and as having its primary stimulatory effect on islet β cell insulin secretion, there have been some reports of its expression in PP cells and α cells; see. e.g., Ahlkvist et al., 2013 Endocrine Connections 2:69-78; Flock et al., 2011 Endocrinology 152:374-383; and Sakamoto et al., 2006 Biochem Biophys Res Commun 351:474-480. Efforts related to GPR119 agonism have been primarily aimed at alleviating hyperglycemia in type 2 diabetic patients.

### SUMMARY OF THE INVENTION

The present invention is based on Applicants' surprising finding that GPR119 agonists directly increase glucagon secretion from the α-cells of the pancreatic islets specifically in the context of hypoglycemia. While some studies have suggested an effect of GPR119 agonists on plasma glucagon in select circumstances and animal models (see. e.g., Ahlkvist et al., 2013 Endocrine Connections 2:69-78; Flock et al., 2011 Endocrinology 152:374-383), Applicants are not aware of any study that has been systematically carried out in the context of hypoglycemia before the Applicant's study. It is believed that this invention represents a potential novel treatment for hypoglycemia.

The present invention relates to a method of preventing hypoglycemia which comprises administering a pharmaceutical composition comprising a GPR119 agonist compound and a pharmaceutically acceptable carrier to a subject at risk for hypoglycemia. In select embodiments, the subject is being treated with a drug or combination of therapies that reduces glucose. In particular embodiments, the subject is being treated with insulin, an insulin analog, an insulin secretagogue such as a sulfonylurea or a non-sulfonylurea secretagogue compound. In particular embodiments, the subject has a history of hypoglycemia. In particular embodiments, the subject has or has had hypoglycemia unawareness. The present invention further relates to a method of treating hypoglycemia which comprises administering a pharmaceutical composition comprising a GPR119 agonist compound and a pharmaceutically acceptable carrier to a subject having a plasma glucose level less that 70 mg/dL. In particular embodiments, the subject has a history of hypoglycemia. In particular embodiments, the subject has or has had hypoglycemia unawareness. In select embodiments, the subject is being treated with a drug or combination of therapies that reduce glucose. In particular embodiments, the subject is being treated with insulin, an insulin analog, an insulin secretagogue such as a sulfonylurea or a non-sulfonylurea secretagogue compound.

In particular embodiments of the present invention, the plasma glucose levels are increased by 5% or more following administration of the GPR119 agonist. In other embodiments, following administration of the GPR119 agonist, the subject experiences glucagon secretion at a glycemia threshold of at least 10 mg/dL higher than experienced prior to the administration. In particular embodiments, the subject has been diagnosed with Type 1 Diabetes. In other embodiments, the subject has been diagnosed with Type 2 Diabetes.

The present invention further relates to an article of manufacture that comprises a container holding (a) a pharmaceutical composition comprising a GPR119 agonist and a pharmaceutically acceptable carrier; and (b) printed instructions for using the pharmaceutical composition for (i) preventing hypoglycemia in a subject at risk for hypoglycemia; and/or (ii) for treating hypoglycemia in a subject diagnosed as having hypoglycemia.

### BRIEF DESCRIPTION OF THE DRAWINGS

FIGS. 1A-E illustrate how GPR119 is highly expressed in pancreatic α-cells. FIG. 1A is a representative FACS plot showing two subsets (YFP+, 15% and YFP−, 74.9%) of islet cells separated from purified islets of GCG-YFP Tg mice. FIGS. 1B-1E illustrate relative mRNA levels of the indicated genes, measured by TAQMAN RT-PCR, in total pancreata from (i) wildtype (C57BL/6) mice, (ii) GCG-YFP Tg mice, and in (iii) purified islets, (iv) FACS sorted YFP+ cells, and (v) FACS-sorted YFP− cells from GCG-YFP Tg mice, respectively.

FIG. 2 illustrates GPR119 expression in mouse and human pancreatic α (glucagon-expressing) and β (insulin-expressing) islet cells. The white arrows outlined in black indicate co-expression of GPR119 mRNA (stained) in cells that stained with glucagon (top panels) or insulin (bottom panels) protein (targeted by the indicated antibodies, respectively). Solid black arrows identify cells expressing GPR119 mRNA but with no glucagon or insulin expression.

FIG. 3 illustrates GPR119 expression in both α and β cells of rhesus monkey pancreatic islet cells. Monkey pancreata were immunostained for glucagon (top panels, dark broad staining), or insulin (bottom panels, dark broad staining), and were con-currently subjected to in situ hybridization of GPR119 mRNA (dark dots). Note that GPR119 mRNA (black arrows) is co-expressed in glucagon or insulin positive cells. The immunostaining was done with an anti-glucagon antibody for the α cells and an anti-insulin antibody for the β cells.

FIGS. 4A-B illustrate a pancreatic perfusion assay in Wistar Han rats, and insulin (FIG. 4A) and glucagon (FIG. 4B) release measurements. As shown (lines with solid circles), a basal level of insulin (left panel) and a fair amount of glucagon (right panel) were secreted from pancreas when perfused with low glucose (6 mM glucose). Once the pancreas was infused with high glucose (12 mM glucose), pancreas insulin secretion was dramatically induced, accompanied with repressed glucagon release. Application of GPR119 agonist (500 nM Compound A, lines with open circles) enhanced both basal and glucose-stimulated pancreas insulin secretion (left panel). In addition to this effect, note that compound A elevated basal glucagon release, but did not affect glucose-repressed glucagon secretion (right panel) from pancreas.

FIG. 5 illustrates a scheme and time-course depiction of the hyperinsulinemic-hypoglycemia clamp study performed in rats as described in EXAMPLE 4. Rats were recovered from surgery stress and pre-treated with vehicle, GLIBENCLAMIDE (5 mpk), or GPR119 agonist Compound B (10 mpk) 60 minutes prior to initiation of the clamp. From time zero, rats received an insulin bolus injection initially, then were simultaneously infused with insulin at a steady rate and glucose at an adjusted rate based on the plasma glucose level measured at every 10 minutes. Vertical bars indicate the time points when the blood specimens were collected for measurements of insulin, C-peptide, glucagon, and epinephrine/norepinephrine (0 min, 30 min, 60 min, 90 min and 120 min).

FIGS. 6A-B illustrate results with Compound B from the hyperinsulinemic-hypoglycemia clamp study of EXAMPLE 4. Rat plasma glucose levels (FIG. 6A) were monitored periodically, and the glucose infusion rate (FIG. 6B) was adjusted so that the blood glucose level would be maintained at 50 mg/dl.

FIGS. 7A-D illustrate measurements of blood glucagon (FIGS. 7A-B), epinephrine (FIG. 7C), and c-peptide (FIG. 7D) levels in rats received from the clamp study of EXAMPLE 4 using Compound B.

FIGS. 8A-L illustrate measurements of blood glucagon levels in rats received from the clamp study of EXAMPLE 4 using Compounds C, F, H, I, M and V.

### DETAILED DESCRIPTION OF THE INVENTION

The present invention relates to a method of preventing hypoglycemia which comprises administering a pharmaceutical composition comprising a GPR119 agonist compound and a pharmaceutically acceptable carrier to a subject at risk for hypoglycemia. GPR119 agonists act as endogenous ligands for the G protein coupled receptor GPR119. In particular embodiments, the GPR119 agonist is a small molecule compound, or in other words a low molecular weight organic compound. A small molecule compound is typically less than 800 Daltons.

Plasma glucose levels in a healthy subject under normal circumstances are maintained in a narrow range, 72-144 mg/dl (4.0-8.0 mmol). When plasma glucose levels start to fall below normal physiological levels (less than 70 mg/dl), the subject will start to experience the initial warning signs of hypoglycemia. Thus, for purposes herein, hypoglycemia, is said to be present in a subject when the subject has a plasma glucose level of less than 70 mg/dl. In distinct embodiments, the subject has a plasma glucose level of less than or equal to 65 mg/dl, less than or equal to 60 mg/dl, less than or equal to 55 mg/dl; less than or equal to 50 mg/dl, or less than or equal to 45 mg/dl.

In particular embodiments, the present invention relates to methods of preventing hypoglycemia described herein which comprise administering a pharmaceutical composition comprising a GPR119 agonist compound and a pharmaceutically acceptable carrier to a subject with a history of hypoglycemia, i.e., has had one or more episodes of hypoglycemia. In particular embodiments, the subject has had at least one prior episode within the last year, within the last 6 months, and/or within the last month. In specific embodiments, the subject has had at least 5 prior episodes of hypoglycemia or at least 2 prior episodes of hypoglycemia. In specific embodiments, the subject has had an episode of severe hypoglycemia that required medical assistance or hospitalization. In particular embodiments, the subject has had or is having a seizure. In particular embodiments, the subject has had at least one seizure within the last year, within the last 6 months, and/or within the last month. In particular embodiments, the subject has or has had hypoglycemia unawareness. In particular embodiments, the subject has had at least one prior episode of hypoglycemia unawareness within the last year, within the last 6 months, and/or within the last month. In specific embodiments, the subject has had at least 5 prior episodes of hypoglycemia unawareness or at least 2 prior episodes of hypoglycemia unawareness.

In select embodiments, the subject is being treated with a drug (or medicinal agent) or combination of drugs that reduces glucose. In specific embodiments, the drug or combination of drug therapies reduces plasma glucose levels to below 70 mg/dL (or 60 mg/dL, 55 mg/dL or 50 mg/dL in particular embodiments thereof). In particular embodiments, the present invention relates to methods of preventing hypoglycemia as described herein where the subject is being treated with insulin or an insulin analog. An insulin analog is an altered form of insulin modified to improve some characteristic such as absorption, distribution, metabolism, and excretion, but still able to similarly effect glycemic control. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In other embodiments, the subject is being treated with an insulin secretagogue. An insulin secretagogue is a substance that causes the secretion of insulin. Sulfonylureas are insulin secretagogues. In particular embodiments, the subject is being treated with a sulfonylurea compound which is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In particular embodiments, the insulin secretagogue is a non-sulfonylurea secretagogue. In specific embodiments, the non-sulfonylurea secretagogue is selected from Repaglinide or Nateglinide. In other embodiments, the subject is being treated with a combination of agents, which combination reduces plasma glucose levels to below 70 mg/dL (or 60 mg/dL, 55 mg/dL or 50 mg/dL in particular embodiments thereof). In particular embodiments, the combination is IDegLira.

In particular embodiments, the present invention relates to methods of preventing hypoglycemia as described herein where the subject is able to maintain plasma glucose levels at or above 70 mg/dl when treated with GPR119 agonist. In particular embodiments, a subject being treated with GPR119 agonist is able to maintain plasma glucose levels at or above 70 mg/dl for a period of 1 week or more, one month or more, four months or more, or six months or more. In particular embodiments, the plasma glucose levels in the subject being treated with GPR119 agonist are increased by 5% or more from the levels prior to treatment. In specific embodiments, the plasma glucose levels are increased by 10% or more from the levels prior to treatment. In specific embodiments, the plasma glucose levels in the subject being treated with GPR119 agonist are increased by at least 5 mg/dl, 10 mg/dl, 15 mg/dl, 20 mg/dl, 25 mg/dl, 30 mg/dl, 35 mg/dl, 40 mg/dl, 45 mg/dl, or 50 mg/dl.

In particular embodiments, the present invention relates to methods of preventing hypoglycemia as described herein where the subject experiences a greater than 5% increase in glucagon secretion. In specific embodiments, glucagon secretion is increased by 10% or more, 20% or more, or 40% more from the levels prior to treatment. In other embodiments, following administration of the GPR119 agonist, the subject experiences glucagon secretion at a glycemia threshold of at least 10 mg/dL higher than experienced prior to the administration. In a specific embodiment thereof, the subject would experience glucagon secretion at 65 mg/dl rather than 55 mg/dl.

In particular embodiments, the present invention relates to methods of preventing hypoglycemia as described herein where the subject has been diagnosed with Type 1 Diabetes. In other embodiments, the present invention relates to methods of preventing hypoglycemia as described herein where the subject has been diagnosed with Type 2 Diabetes. In particular embodiments, the present invention relates to methods of preventing hypoglycemia as described herein where the subject has been diagnosed with latent autoimmune diabetes in adults (LADA). In particular embodiments, the present invention relates to methods of preventing hypoglycemia as described herein where the subject has been diagnosed with cystic fibrosis-related diabetes. In particular embodiments, the present invention relates to methods of preventing hypoglycemia as described herein where the subject has been diagnosed with diabetes secondary to pancreatectomy. In other embodiments, the present invention relates to methods of preventing hypoglycemia resulting from gastric bypass, reactive or post prandial hypoglycemia, an insulinoma, insulin- or insulin-like growth factor (IGF)—secreting tumors, paraneoplastic conditions associated with hypoglycemia, or other condition associated with hypoglycemia.

The present invention further relates to a method of treating hypoglycemia which comprises administering a pharmaceutical composition comprising a GPR119 agonist compound and a pharmaceutically acceptable carrier to a subject having a plasma glucose level of less than 70 mg/dl. In particular embodiments, the subject has a plasma glucose level of less than or equal to 65 mg/dl, less than or equal to 60 mg/dl, less than or equal to 55 mg/dl; less than or equal to 50 mg/dl, or less than or equal to 45 mg/dl.

In particular embodiments, the present invention relates to methods of treating hypoglycemia described herein which comprise administering a pharmaceutical composition comprising a GPR119 agonist compound and a pharmaceutically acceptable carrier to a subject with a history of hypoglycemia. In particular embodiments, the subject has had at least one prior episode within the last year, within the last 6 months, and/or within the last month. In specific embodiments, the subject has had at least 5 prior episodes of hypoglycemia or at least 2 prior episodes of hypoglycemia. In specific embodiments, the subject has had an episode of severe hypoglycemia that required medical assistance or hospitalization. In particular embodiments, the subject has had or is having a seizure. In particular embodiments, the subject has had at least one seizure within the last year, within the last 6 months, and/or within the last month. In particular embodiments, the subject has or has had hypoglycemia unawareness. In particular embodiments, the subject has had at least one prior episode of hypoglycemia unawareness within the last year, within the last 6 months, and/or within the last month. In specific embodiments, the subject has had at least 5 prior episodes of hypoglycemia unawareness or at least 2 prior episodes of hypoglycemia unawareness.

In select embodiments, the subject is being treated with a drug (or medicinal agent) or combination of drugs that reduces glucose. In specific embodiments, the drug or combination of drug therapies reduces plasma glucose levels to below 70 mg/dL (or 60 mg/dL, 55 mg/dL or 50 mg/dL in particular embodiments thereof). In particular embodiments, the present invention relates to methods of treating hypoglycemia as described herein where the subject is being treated with insulin or an insulin analog. An insulin analog is an altered form of insulin modified to improve some characteristic such as absorption, distribution, metabolism, and excretion, but still able to similarly effect glycemic control. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In other embodiments, the subject is being treated with an insulin secretagogue. An insulin secretagogue is a substance that causes the secretion of insulin. Sulfonylureas are insulin secretagogues. In particular embodiments, the subject is being treated with a sulfonylurea compound which is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In particular embodiments, the insulin secretagogue is a non-sulfonylurea secretagogue. In specific embodiments, the non-sulfonylurea secretagogue is selected from Repaglinide or Nateglinide. In other embodiments, the subject is being treated with a combination of agents, which combination reduces plasma glucose levels to below 70 mg/dL (or 60 mg/dL, 55 mg/dL or 50 mg/dL in particular embodiments thereof). In particular embodiments, the combination is IDegLira.

In particular embodiments, the present invention relates to methods of treating hypoglycemia as described herein where the subject is able to maintain plasma glucose levels at or above 70 mg/dl when treated with GPR119 agonist. In particular embodiments, a subject being treated with GPR119 agonist is able to maintain plasma glucose levels at or above 70 mg/dl for a period of 1 week or more, one month or more, four months or more, or six months or more. In particular embodiments, the plasma glucose levels in the subject being treated with GPR119 agonist are increased by 5% or more from the levels prior to treatment. In specific embodiments, the plasma glucose levels are increased by 10% or more from the levels prior to treatment. In specific embodiments, the plasma glucose levels in the subject being treated with GPR119 agonist are increased by at least 5 mg/dl, 10 mg/dl, 15 mg/dl, 20 mg/dl, 25 mg/dl, 30 mg/dl, 35 mg/dl, 40 mg/dl, 45 mg/dl, or 50 mg/dl.

In particular embodiments, the present invention relates to methods of treating hypoglycemia as described herein where the subjectexperiences a greater than 5% increase in glucagon secretion. In specific embodiments, glucagon secretion is increased by 10% or more, 20% or more, or 40% more from the levels prior to treatment. In other embodiments, following administration of the GPR119 agonist, the subject experiences glucagon secretion at a glycemia threshold of at least 10 mg/dL higher than experienced prior to the administration. In a specific embodiment thereof, the subject would experience glucagon secretion at 65 mg/dl rather than 55 mg/dl.

In particular embodiments, the present invention relates to methods of treating hypoglycemia as described herein where the subject has been diagnosed with Type 1 Diabetes. In other embodiments, the present invention relates to methods of treating hypoglycemia as described herein where the subject has been diagnosed with Type 2 Diabetes. In particular embodiments, the present invention relates to methods of treating hypoglycemia as described herein where the subject has been diagnosed with latent autoimmune diabetes in adults (LADA). In particular embodiments, the present invention relates to methods of treating hypoglycemia as described herein where the subject has been diagnosed with cystic fibrosis-related diabetes. In particular embodiments, the present invention relates to methods of treating hypoglycemia as described herein where the subject has been diagnosed with diabetes secondary to pancreatectomy. In other embodiments, the present invention relates to methods of treating hypoglycemia resulting from gastric bypass, reactive or post prandial hypoglycemia, an insulinoma, insulin- or insulin-like growth factor (IGF)—secreting tumors, paraneoplastic conditions associated with hypoglycemia, or other condition associated with hypoglycemia.

In particular embodiments, the present invention relates to methods of preventing or treating hypoglycemia as described herein wherein the GPR119 agonist is a compound disclosed or described in one of the following publications: WO 2007/003962, WO 2008/070692, WO 2008/083238, WO 2008/097428; WO 2008/109702; WO 2009/038974; WO 2009/055331, WO 2009/105715; WO 2009/105717; WO 2009/105722; WO 2009/126535; WO 2009/129036, WO 2010/006191; WO 2010/149684; WO 2010/149685; WO 2011/014520; WO 2011/044001; WO 2011/127106, WO 2011/128394; WO 2011/128395; WO 2012/123449; WO2012/138845, WO 2012/168315; WO 2012/173917, WO 2013/074388, WO 2014/016191; WO 2014/019967; WO 2014/037327; WO 2014/052619, Bioorg. Med. Chem. Lett. 2014, 24, 4332-4335, ACS Medicinal Chemistry Letters 2015, 6(8), 936-941, or J. Med. Chem. 2014, 57, 7499-7508; each of which is incorporated herein by reference.

In particular embodiments, the present invention relates to methods of preventing or treating hypoglycemia as described herein wherein the GPR119 agonist is a compound disclosed or described in one of the following publications: JP2013063913, WO2005007658, WO2005061489, WO2007116229, WO2009106561, WO2009106565, WO2009125434, WO2009126535, WO2009141238, WO2010084512, WO2010123018, WO2010146605, WO2010048149, WO2010088518, WO2011005929, WO2011014520, WO2011025006, WO2011078306, WO2011093501, WO2011127051, WO2012040279, WO2012046249, WO2012046792, WO2012098217, WO2012111995, WO2012117996, WO2012135570, WO2012145603, WO2012145361, WO2012170702, WO2013167514, or WO2013186109; each of which is incorporated herein by reference.

In select embodiments, the present invention relates to methods of preventing or treating hypoglycemia as described herein wherein the GPR119 agonist is the compound known as: PSN821, GSK1292263, MBX-2982, DS-8500, DS-8500a, LEZ763, APD668, APD597, ZYG-19, BMS-903452, NN, HD0471042, JRP206, HM47000, KR69318, GSK2041706, DA1241, ZY7334, R05212651, LC340767, NIP361, LC34AD3, RP9056, CZMD001, or AR228407.

In particular embodiments, the present invention relates to methods of preventing or treating hypoglycemia as described herein wherein the GPR119 agonist is selected from the following table of compounds:

TABLE 1

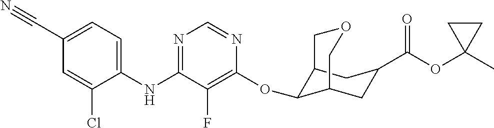 [SMILES: CC1(OC(=O)C2CC3COCC(C2)C3Oc2ncnc(Nc3ccc(C#N)cc3Cl)c2F)CC1]

as disclosed and described in WO 2009/055331
incorporated herein by reference,
see, e.g., Example 251;
and referred to herein as Compound A

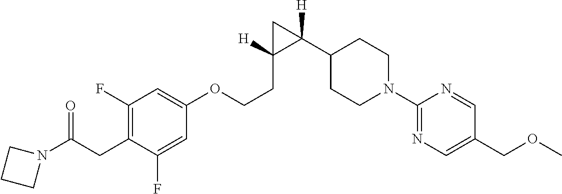 [SMILES: COCc1cnc(N2CCC([C@H]3C[C@H]3CCOc3cc(F)c(CC(=O)N4CCC4)c(F)c3)CC2)nc1]

as disclosed and described in WO 2013/074388
incorporated herein by reference,
see, e.g., Example 219;
and referred to herein as Compound B

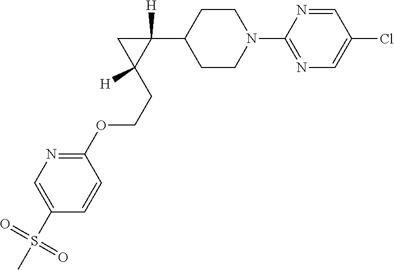 [SMILES: CS(=O)(=O)c1ccc(OCC[C@@H]2C[C@@H]2C2CCN(c3ncc(Cl)cn3)CC2)nc1]

as disclosed and described in WO 2009/129036
incorporated herein by reference,
see, e.g., Example 6;
and referred to herein as Compound C

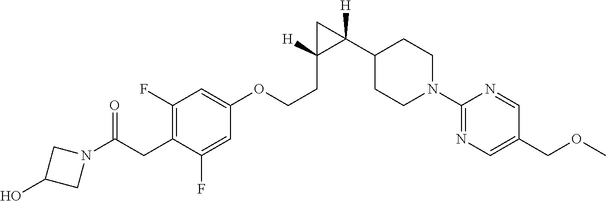 [SMILES: COCc1cnc(N2CCC([C@H]3C[C@H]3CCOc3cc(F)c(CC(=O)N4CC(O)C4)c(F)c3)CC2)nc1]

as disclosed and described in WO 2013/074388
incorporated herein by reference,
see, e.g., Example 218;
and referred to herein as Compound D

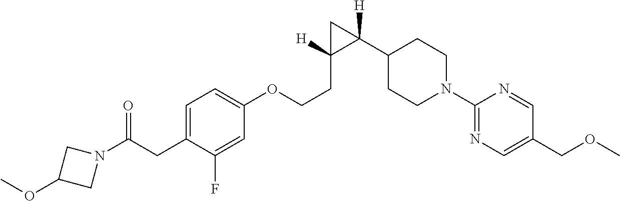

as disclosed and described in WO 2013/074388
incorporated herein by reference,
see, e.g., Example 45;
and referred to herein as Compound E

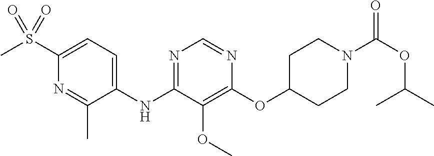 [SMILES: COc1c(Nc2ccc(S(C)(=O)=O)nc2C)ncnc1OC1CCN(C(=O)OC(C)C)CC1]

as disclosed and described in Bioorg. Med. Chem.
Lett. 2014, 24, 4332-4335 and
WO 2006/83491 (see, e.g., Compound 84)
incorporated herein by reference,
and referred to herein as Compound F

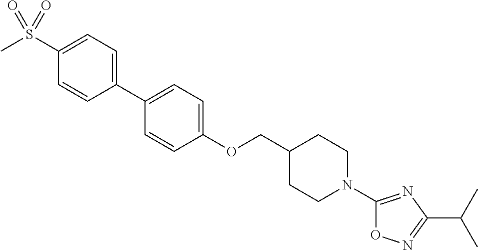 [SMILES: CC(C)c1noc(N2CCC(COc3ccc(-c4ccc(S(C)(=O)=O)cc4)cc3)CC2)n1]

as disclosed and described in WO 2008/070692
incorporated herein by reference,
see, e.g., Example 100;
and referred to herein as Compound G

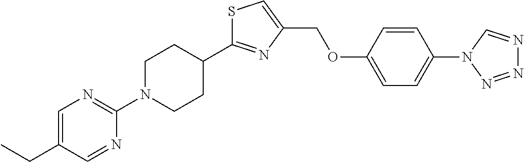 [SMILES: CCc1cnc(N2CCC(c3nc(COc4ccc(-n5cnnn5)cc4)cs3)CC2)nc1]

as disclosed and described in WO 2008/083238
incorporated herein by reference,
see, e.g., Example 52;
and referred to herein as Compound H

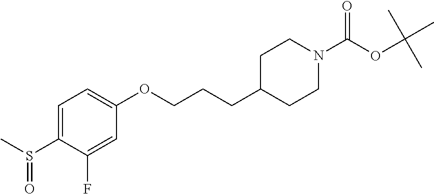 [SMILES: CS(=O)c1ccc(OCCCC2CCN(C(=O)OC(C)(C)C)CC2)cc1F]

as disclosed and described in WO 2007/003962
incorporated herein by reference,
see, e.g., Example 148;
and referred to herein as Compound I

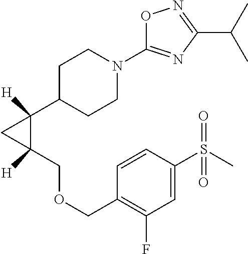 [SMILES: CC(C)c1noc(N2CCC([C@H]3C[C@H]3COCc3ccc(S(C)(=O)=O)cc3F)CC2)n1]

as disclosed and described in WO 2012/173917
incorporated herein by reference,
see, e.g., Example 78;
and referred to herein as Compound J

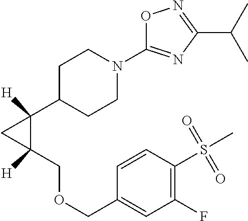 [SMILES: CC(C)c1noc(N2CCC([C@H]3C[C@H]3COCc3ccc(S(C)(=O)=O)c(F)c3)CC2)n1]

as disclosed and described in WO 2012/173917
incorporated herein by reference,
see, e.g., Example 35;
and referred to herein as Compound K

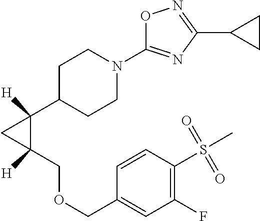 [SMILES: CS(=O)(=O)c1ccc(COC[C@@H]2C[C@@H]2C2CCN(c3nc(C4CC4)no3)CC2)cc1F]

as disclosed and described in WO 2012/173917
incorporated herein by reference,
see, e.g., Example 37;
and referred to herein as Compound L

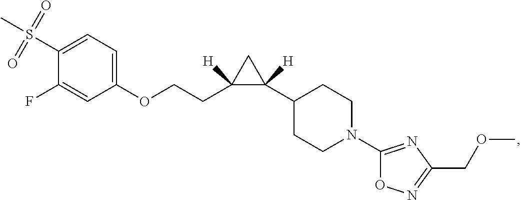

as disclosed and described in W02012/138845
incorporated herein by reference,
see, e.g., Example 222;
and referred to herein as Compound M

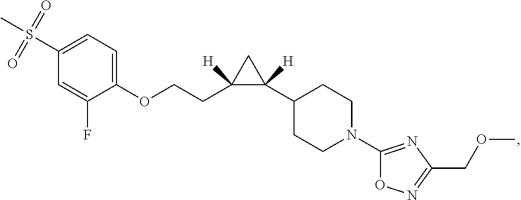

as disclosed and described in W02012/138845
incorporated herein by reference,
see, e.g., Example 220;
and referred to herein as Compound N

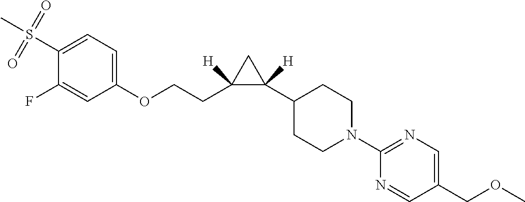

as disclosed and described in
ACS Medicinal Chemistry Letters,
2015, 6(8), 936-941
incorporated herein by reference;
and referred to herein as Compound O

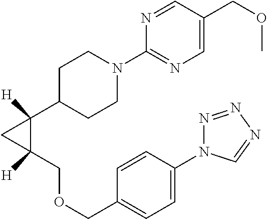 [SMILES: COCc1cnc(N2CCC([C@H]3C[C@H]3COCc3ccc(-n4cnnn4)cc3)CC2)nc1]

as disclosed and described in WO 2012/173917
incorporated herein by reference,
see, e.g., Example 132;
and referred to herein as Compound P

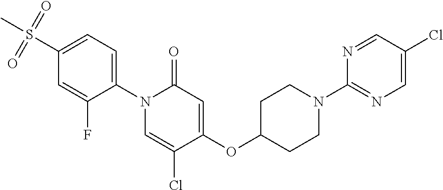 [SMILES: CS(=O)(=O)c1ccc(-n2cc(Cl)c(OC3CCN(c4ncc(Cl)cn4)CC3)cc2=O)c(F)c1]

as disclosed and described in
J. Med. Chem. 2014, 57, 7499-7508 and
WO 2011/127106 incorporated herein by reference,
see, e.g., Example 5;
and referred to herein as Compound Q
or a pharmaceutically acceptable salt thereof.

In particular embodiments, the present invention relates to methods of preventing or treating hypoglycemia as described herein wherein the GPR119 agonist is selected from the following table of compounds:

TABLE 2

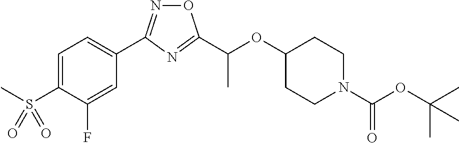 [SMILES: CC(OC1CCN(C(=O)OC(C)(C)C)CC1)c1nc(-c2ccc(S(C)(=O)=O)c(F)c2)no1]

as disclosed and described in WO 2007/116229
incorporated herein by reference,
see, e.g., Examples 23, 24, and 45;
and referred to herein as Compound R

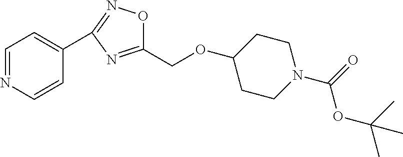 [SMILES: CC(C)(C)OC(=O)N1CCC(OCc2nc(-c3ccncc3)no2)CC1]

as disclosed and described in WO 2005/061489
incorporated herein by reference,
see e.g., Example 1;
and referred to herein as Compound S

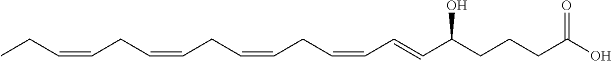 [SMILES: CC/C=C\C/C=C\C/C=C\C/C=C\C=C\[C@@H](O)CCCC(=O)O]

as disclosed in Kogure et al., 2011 Biochem Biophy
Res Comm 416:58-63; JP 2013063913 and Primdahl
et al., 2016 Chem Phys Lipids 196: 1-4;
and referred to herein as Compound T

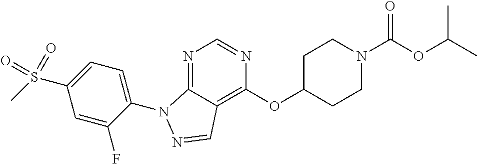 [SMILES: CC(C)OC(=O)N1CCC(Oc2ncnc3c2cnn3-c2ccc(S(C)(=O)=O)cc2F)CC1]

as disclosed and described in WO 2005/007658
incorporated herein by reference,
see, e.g., Compound A129;
and referred to herein as Compound U

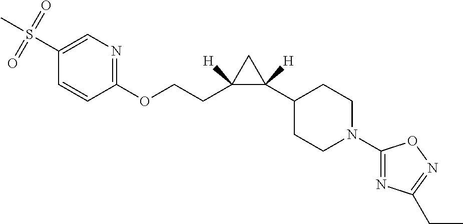 [SMILES: CCc1noc(N2CCC([C@H]3C[C@H]3CCOc3ccc(S(C)(=O)=O)cn3)CC2)n1]

as disclosed and described in WO 2012/138845
incorporated herein by reference,
see, e.g., Example 214;
and referred to herein as Compound V
or a pharmaceutically acceptable salt thereof.

In specific embodiments, the GPR119 agonist of use in the present invention may include hydrates, solvates or polymorphic forms of the GPR119 agonist compounds described herein.

The present invention also relates to a combination of (a) a GPR119 agonist (b) a drug(s) that reduces plasma glucose levels to below 70 mg/dL (or 60 mg/dL, 55 mg/dL or 50 mg/dL in particular embodiments thereof), and (c) a pharmaceutically acceptable carrier. In particular embodiments, the present invention relates to a combination of (a) a GPR119 agonist (b) a molecule selected from (i) insulin, (ii) an insulin analog, or (iii) an insulin secretagogue, and (c) a pharmaceutically acceptable carrier. In particular embodiments, the present invention relates to a combination of a GPR119 agonist, insulin and a pharmaceutically acceptable carrier. In specific embodiments, the present invention relates to a combination of GPR119 agonist, an insulin analog and a pharmaceutically acceptable carrier. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In specific embodiments, the present invention relates to a combination of GPR119 agonist, an insulin secretagogue and a pharmaceutically acceptable carrier. In specific embodiments, the present invention relates to a combination of GPR119 agonist, a sulfonylurea compound and a pharmaceutically acceptable carrier. In particular embodiments, the sulfonylurea compound is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In specific embodiments, the present invention relates to a combination of GPR119 agonist, a non-sulfonylurea secretagogue and a pharmaceutically acceptable carrier. In specific embodiments, the non-sulfonylurea secretagogue is selected from Repaglinide or Nateglinide. In specific embodiments, the present invention relates to a combination of: GPR119 agonist, a combination of agents which reduces plasma glucose levels to below 70 mg/dL (or 60 mg/dL, 55 mg/dL or 50 mg/dL in particular embodiments thereof), and a pharmaceutically acceptable carrier. In particular embodiments, the combination which reduces plasma glucose levels is IDegLira. The present invention further relates to the use of one of the above-described combinations for the treatment of type 2 diabetes.

As used herein “preventing” and alternate versions thereof such as “prevention” means the avoidance of plasma glucose level of less than 70 mg/dl in a subject. As used herein “treating” and alternate versions thereof such as “treatment” means the alleviation of symptoms and effects characteristic of hypoglycemia in a subject. Throughout the present application, the terms patient, individual and subject will be used interchangeably and mean the same thing—an individual receiving GPR119 agonist for the purposes described herein.

As used herein the terms “subject”, “individual” or “patient” means in addition to primates, such as humans, a variety of other mammals that can be treated according to the method of the present invention; including, but not limited to, cows, sheep, goats, horses, dogs, cats, guinea pigs, rats or other bovine, ovine, equine, canine, feline, rodent or murine species. In particular embodiments, the subject, individual or patient is a human. In specific embodiments, the subject, individual or patient is an adult human.

In specific embodiments of the present invention, prior to administration of the GPR119 agonist, the plasma glucose levels are measured. Means for measuring plasma glucose levels are known and described in the art. When specific amounts are provided, the amount of plasma glucose intended is that which would be obtained using the hexokinase procedures as described in Glampletro et al., 1982 Clin. Chem. 2405-2407—if that measurement technique had been utilized.

The present invention further relates to the use of a pharmaceutical composition comprising a GPR119 agonist alone or in combination with a drug(s) that reduces plasma glucose levels to below 70 mg/dL (or 60 mg/dL, 55 mg/dL or 50 mg/dL in particular embodiments thereof), and a pharmaceutically acceptable carrier for the manufacture of a medicament for preventing or treating hypoglycemia in a patient as described and disclosed herein. In specific embodiments, the pharmaceutical composition comprises a GPR119 agonist alone or in combination with insulin, an insulin analog or an insulin secretagogue, and a pharmaceutically acceptable carrier. In specific embodiments, the pharmaceutical composition comprises a GPR119 agonist and a pharmaceutically acceptable carrier. In particular embodiments, the pharmaceutical composition comprises a GPR119 agonist, insulin, and a pharmaceutically acceptable carrier and is for the manufacture of a medicament for treating diabetes. In specific embodiments, the pharmaceutical composition comprises a GPR119 agonist, an insulin analog and a pharmaceutically acceptable carrier and is for the manufacture of a medicament for treating diabetes. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In other embodiments, the pharmaceutical composition comprises a GPR1219 agonist, an insulin secretagogue and a pharmaceutically acceptable carrier and is for the manufacture of a medicament for treating diabetes. In specific embodiments, the pharmaceutical composition comprises a GPR119 agonist, an insulin secretagogue selected from carbutamide, tolbutamide, tolazamide, chlorpropamide, acetohexamide, glyburide (also known as glibenclamide), glipizide, gliclazide, glibomuride, glipquidone, glisoxepide, glyclopyramide and glimepiride, and a pharmaceutically acceptable carrier and is for the manufacture of a medicament for use in treating diabetes. In specific embodiments, the pharmaceutical composition comprises a GPR119 agonist, an insulin secretagogue selected from Repaglinide or Nateglinide, and a pharmaceutically acceptable carrier and is for the manufacture of a medicament for use in treating diabetes. In specific embodiments, the pharmaceutical composition comprises a GPR119 agonist, a combination of agents which reduces plasma glucose levels to below 70 mg/dL (or 60 mg/dL, 55 mg/dL or 50 mg/dL in particular embodiments thereof), and a pharmaceutically acceptable carrier and is for the manufacture of a medicament for use in treating diabetes. In specific embodiments, the pharmaceutical composition comprises a GPR119 agonist, IDegLira, and a pharmaceutically acceptable carrier and is for the manufacture of a medicament for use in treating diabetes. In specific embodiments, the above-described pharmaceutical compositions are for treating hypoglycemia. In other embodiments, the above-described pharmaceutical compositions are for use in preventing hypoglycemia. In other embodiments, the above-described pharmaceutical compositions are for use in treating hypoglycemia.

The present invention further relates to an article of manufacture that comprises a container holding: (a) a pharmaceutical composition comprising a GPR119 agonist alone or in combination with a drug(s) that reduces plasma glucose levels to below 70 mg/dL (or 60 mg/dL, 55 mg/dL or 50 mg/dL in particular embodiments thereof), and a pharmaceutically acceptable carrier, and (b) printed instructions for preventing or treating hypoglycemia in a patient as described and disclosed herein. In select embodiments, the present invention relates to an article of manufacture that comprises a container holding: (a) a pharmaceutical composition comprising a GPR119 agonist alone or in combination with insulin, an insulin analog or an insulin secretagogue, and a pharmaceutically acceptable carrier, and (b) printed instructions for preventing or treating hypoglycemia in a patient as described and disclosed herein. In specific embodiments, the printed instructions comprise instructions for treating hypoglycemia. In other embodiments, the printed instructions comprise instructions for preventing hypoglycemia. In specific embodiments, the pharmaceutical composition comprises a GPR119 agonist and a pharmaceutically acceptable carrier. In particular embodiments, the pharmaceutical composition comprises a GPR119 agonist, insulin, and a pharmaceutically acceptable carrier and the printed instructions also comprise instructions for treating diabetes. In specific embodiments, the pharmaceutical composition comprises a GPR119 agonist, an insulin analog and a pharmaceutically acceptable carrier and the printed instructions also comprise instructions for treating diabetes. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NNI218. In other embodiments, the pharmaceutical composition comprises a GPR119 agonist, an insulin secretagogue and a pharmaceutically acceptable carrier and the printed instructions also comprise instructions for treating diabetes. In specific embodiments, the pharmaceutical composition comprises a GPR119 agonist, an insulin secretagogue selected from carbutamide, tolbutamide, tolazamide, chlorpropamide, acetohexamide, glyburide (also known as glibenclamide), glipizide, gliclazide, glibomuride, glipquidone, glisoxepide, glyclopyramide and glimepiride, and a pharmaceutically acceptable carrier and the printed instructions also comprise instructions for treating diabetes. In specific embodiments, the pharmaceutical composition comprises a GPR119 agonist, an insulin secretagogue selected from Repaglinide or Nateglinide, and a pharmaceutically acceptable carrier and the printed instructions also comprise instructions for treating diabetes. In specific embodiments, the pharmaceutical composition comprises a GPR119 agonist, a combination of agents which reduces plasma glucose levels to below 70 mg/dL (or 60 mg/dL, 55 mg/dL or 50 mg/dL in particular embodiments thereof), and a pharmaceutically acceptable carrier and the printed instructions also comprise instructions for treating diabetes. In specific embodiments, the combination of agents that reduces plasma glucose levels to below 70 mg/dL (or 60 mg/dL, 55 mg/dL or 50 mg/dL in particular embodiments) is IDegLira.

The present invention derives from Applicants' findings that GPR119 agonists promote glucagon release from pancreatic islets only in the hypoglycemic state, but has no effect on glucagon secretion at euglycemic or hyperglycemic conditions (e.g. Example 3&4). This finding is significant for the prevention and treatment of hypoglycemia in patients at risk for hypoglycemia. In combination with a drug or combination of drugs that reduces plasma glucose levels to below 70 mg/dL (or 60 mg/dL, 55 mg/dL or 50 mg/dL in particular embodiments), including but not limited to insulin, an insulin analog, or an insulin secretagogue, treatment with GPR119 agonist will provide a fine modulation on the control of and prevent any consequent decrease of plasma glucose levels to progressively concerning levels below 70 mg/dL, 60 mg/dL or 50 mg/dL which are associated with hypoglycemia and its associated clinical symptoms as compared to use of insulin, an insulin analog, insulin secretagogue or other agent reducing plasma glucose alone. This finding opens up new therapeutic possibilities in the treatment and prevention of hypoglycemia in patients at risk for hypoglycemia including patients being treated for type 1 or type 2 diabetes mellitus and related disease conditions.

In use for therapy, the present invention contemplates that the GPR119 agonist and the drug or combination of drugs that reduces plasma glucose levels to below 70 mg/dL (or 60 mg/dL, 55 mg/dL or 50 mg/dL in particular embodiments) are administered in therapeutically effective amounts. A “therapeutically effective amount” is the dose or amount that produces a therapeutic response or desired effect in a subject or group of individuals. The therapeutic response or desired effect for the GPR119 compound is defined herein as the prevention of hypoglycemia in a patient at risk for hypoglycemia. The therapeutic response or desired effect for the insulin, insulin analog or insulin secretagogue is defined herein as the treatment of diabetes and in particular type 2 diabetes or type 1 diabetes. In particular embodiments, the subject has a history of hypoglycemia. In particular embodiments, the subject has or has had hypoglycemia unawareness. In particular embodiments, the therapeutic response or desired effect for the GPR119 agonist is preventing hypoglycemia such that the subject is able to maintain plasma glucose levels at or above 70 mg/dl. In particular embodiments, a subject being treated with GPR119 agonist is able to maintain plasma glucose levels at or above 70 mg/dl for a period of 1 week or more, one month or more, four months or more, or six months or more. In particular embodiments, the plasma glucose levels in the subject being treated with GPR119 agonist are increased by 5% or more from the levels prior to treatment. In specific embodiments, the plasma glucose levels are increased by 10% or more from the levels prior to treatment. In specific embodiments, the plasma glucose levels in the subject being treated with GPR119 agonist are increased by at least 5 mg/dl, 10 mg/dl, 15 mg/dl, 20 mg/dl, 25 mg/dl, 30 mg/dl, 35 mg/dl, 40 mg/dl, 45 mg/dl, or 50 mg/dl. In particular embodiments, the therapeutic response or desired effect is prevention of hypoglycemia wherein the subject experiences a greater than 5% increase in glucagon secretion. In specific embodiments, glucagon secretion is increased by 10% or more, 20% or more, or 40% more from the levels prior to treatment. In other embodiments, following administration of the GPR119 agonist, the subject experiences glucagon secretion at a glycemia threshold of at least 10 mg/dL higher than experienced prior to the administration. In a specific embodiment thereof, the subject would experience glucagon secretion at 65 mg/dl rather than 55 mg/dl. The GPR119 agonist can be administered before, following, simultaneously or sequentially with the drug or combination of drugs that reduces plasma glucose levels to below 70 mg/dL (or 60 mg/dL, 55 mg/dL or 50 mg/dL in particular embodiments).

The dose, prophylactic or therapeutic, of the GPR119 agonist or the drug or combination of drugs that reduces plasma glucose levels to below 70 mg/dL (or 60 mg/dL, 55 mg/dL or 50 mg/dL in particular embodiments) will, of course, vary with the nature or severity of the condition to be treated, the particular compound selected and its route of administration. It may also vary according to the age, weight and response of the individual patient. In general, the daily dose range for the individual compounds lies within the range of from about 0.001 mg to about 100 mg per kg, in additional embodiments about 0.01 mg to about 50 mg per kg, and in further embodiments 0.1 to 10 mg per kg, in single or divided doses. It may be necessary to use dosages outside of these limits in some cases. A therapeutically effective amount or a therapeutic or prophylactic dose or terms of similar meaning appearing throughout the application addressing the amount of the compound to be used refer to the dosage ranges provided, taking into account any necessary variation outside of these ranges. These amounts can be readily determined by the skilled physician.

Representative dosages of the individual GPR119 agonist or insulin, insulin analog, or insulin secretagogue for adult humans range from about 0.1 mg to about 1.0 g per day, preferably about 1 mg to about 500 mg, in single or divided doses. Examples of suitable dosages per day include 0.1 mg, 1 mg, 2 mg, 5 mg, 10 mg, 20 mg, 40 mg, 50 mg, 75 mg, 100 mg, 150 mg, 200 mg, 250 mg, 500 mg, 1000 mg and similar such doses.

In particular embodiments, the present invention relates to methods and compositions herein where the dosage of the GPR119 agonist and/or drug(s) that reduces plasma glucose levels to below 70 mg/dL (or 60 mg/dL, 55 mg/dL or 50 mg/dL in particular embodiments) lies within the range of from about 0.001 mg to about 100 mg per kg, in additional embodiments about 0.01 mg to about 50 mg per kg, and in further embodiments 0.1 to 10 mg per kg, in single or divided doses. In particular embodiments, the dosage is in a range of 0.1 mg per kg to 5 mg per kg. In particular embodiments, the dosage of GPR119 agonist and/or drug(s) that reduces plasma glucose levels to below 70 mg/dL (or 60 mg/dL, 55 mg/dL or 50 mg/dL in particular embodiments) is below 0.1 mg per kg. In particular embodiments, the present invention relates to methods and compositions herein where the dosage of GPR119 agonist and/or drug(s) that reduces plasma glucose levels to below 70 mg/dL (or 60 mg/dL, 55 mg/dL or 50 mg/dL in particular embodiments) is any amount in the range of 10-120 mg per day. In specific embodiments, the dosage of GPR119 agonist and/or drug(s) that reduces plasma glucose levels to below 70 mg/dL (or 60 mg/dL, 55 mg/dL or 50 mg/dL in particular embodiments) is 10 mg, 20 mg, 40 mg, 60 mg, 80 mg, 100 mg or 120 mg per day. In specific individual embodiments, the dosage of GPR119 agonist is 10 mg, 20 mg, 40 mg, 60 mg, 80 mg, 100 mg or 120 mg per day. In specific embodiments, the dosage of GPR119 agonist and/or drug(s) that reduces plasma glucose levels to below 70 mg/dL (or 60 mg/dL, 55 mg/dL or 50 mg/dL in particular embodiments) is any amount in the range of 1-100 mg per day. In more specific embodiments, the dosage of GPR119 agonist and/or drug(s) that reduces plasma glucose levels to below 70 mg/dL (or 60 mg/dL, 55 mg/dL or 50 mg/dL in particular embodiments) is any amount in the range of 6-50 mg per day. In specific embodiments, the dosage of GPR119 agonist is any amount in the range of 6-50 mg per day.

In particular embodiments, the present invention relates to methods and compositions herein where (a) the dosage of GPR119 agonist and/or drug(s) that reduces plasma glucose levels to below 70 mg/dL (or 60 mg/dL, 55 mg/dL or 50 mg/dL in particular embodiments) is individually (i) in a range of 0.1 mg per kg to 2 mg per kg; (ii) below 0.1 mg per kg, (iii) in the range of 10-120 mg per day, (iv) 10 mg per day, (v) 20 mg per day, (vi) 40 mg per day, (vii) 60 mg per day. (viii) 80 mg per day, (ix) 100 mg per day, (x) 120 mg per day, (xi) in the range of 1-100 mg per day and/or (xii) in the range of 6-50 mg per day.

When intravenous administration is employed, a representative dosage range is from about 0.001 mg to about 100 mg (preferably from 0.01 mg to about 10 mg) per kg of body weight per day of each of the GPR119 agonist and/or drug(s) that reduces plasma glucose levels to below 70 mg/dL (or 60 mg/dL, 55 mg/dL or 50 mg/dL in particular embodiments) and, in particular embodiments about 0.1 mg to about 10 mg of the compounds per kg of body weight per day.

The GPR119 agonist and/or drug(s) that reduce plasma glucose levels to below 70 mg/dL (or 60 mg/dL, 55 mg/dL or 50 mg/dL in particular embodiments) are used with one or more pharmaceutically acceptable carriers. In particular embodiments, the GPR119 agonist and/or drug(s) that reduces plasma glucose levels to below 70 mg/dL (or 60 mg/dL, 55 mg/dL or 50 mg/dL in particular embodiments) have distinct pharmaceutically acceptable carriers. In specific embodiments, the GPR119 agonist and/or drug(s) that reduces plasma glucose levels to below 70 mg/dL (or 60 mg/dL, 55 mg/dL or 50 mg/dL in particular embodiments) have the same pharmaceutically acceptable carrier. Pharmaceutical compositions of use herein comprise (i) a GPR119 agonist alone or a combination of a GPR119 agonist and a drug (or combination of drugs) that reduces plasma glucose levels to below 70 mg/dL (or 60 mg/dL, 55 mg/dL or 50 mg/dL in particular embodiments), and (ii) at least one pharmaceutically acceptable carrier. In particular embodiments, pharmaceutical compositions of use herein comprise (i) a a combination of a GPR119 agonist and an insulin, insulin analog, or insulin secretagogue, and (ii) at least one pharmaceutically acceptable carrier. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In particular embodiments, the insulin secretagogue is a sulfonylurea compound. In particular embodiments, the insulin secretagogue is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In particular embodiments, the insulin secretagogue is a non-sulfonylurea compound. In particular embodiments, the insulin secretagogue is Repaglinide or Nateglinide. In specific embodiments, the drug (or combination of drugs) that reduces plasma glucose levels to below 70 mg/dL (or 60 mg/dL, 55 mg/dL or 50 mg/dL in particular embodiments) is IDegLira. The term “composition” encompasses a product comprising the active and inert ingredient(s), any pharmaceutically acceptable excipients that make up the carrier, as well as any product which results, directly or indirectly, from the combination, complexation or aggregation of any two or more of the ingredients, or from dissociation of one or more of the ingredients, or from other types of reactions or interactions between ingredients. The composition comprises therapeutically effective amounts of the GPR119 agonist, or both the GPR119 agonist and the drug (or combination of drugs) that reduces plasma glucose levels to below 70 mg/dL (or 60 mg/dL, 55 mg/dL or 50 mg/dL in particular embodiments) as applicable.

Any suitable route of administration may be employed for providing a mammal, especially a human, with an effective dosage of the individual GPR119 agonist and/or drug (or combination of drugs) that reduces plasma glucose levels to below 70 mg/dL (or 60 mg/dL, 55 mg/dL or 50 mg/dL in particular embodiments). For example, oral, rectal, topical, parenteral, ocular, pulmonary, nasal, and the like may be employed. Examples of dosage forms include tablets, troches, dispersions, suspensions, solutions, capsules, creams, ointments, acrosols and the like, with oral tablets being preferred.

In preparing oral compositions, any of the usual pharmaceutical media may be employed, such as, for example, water, glycols, oils, alcohols, flavoring agents, preservatives, coloring agents and the like, in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets. Solid oral preparations are preferred. Because of their ease of administration, tablets and capsules represent the most advantageous oral dosage unit forms. If desired, tablets may be coated by standard aqueous or nonaqueous techniques.

In addition to the common dosage forms set out above, the compounds may also be administered by controlled release means and/or delivery devices. Various controlled release means and/or delivery devices are known in the art.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any acceptable pharmaceutical process. All such methods include the step of combining the active ingredients (either GPR119 agonist or drug (or combination of drugs) that reduces plasma glucose levels to below 70 mg/dL or, in particular embodiments, 60 mg/dL, 55 mg/dL or 50 mg/dL) with the carrier components. In general, the compositions are prepared by uniformly and intimately admixing the active ingredients with a liquid or finely divided solid carrier component, and then, if necessary, manipulating the blend into the desired product form. For example, a tablet may be prepared by compression or molding. Compressed tablets may be prepared by compressing free-flowing powder or granules, containing the actives optionally mixed with one or more excipients, e.g., binders, lubricants, diluents, surfactants and dispersants. Molded tablets may be made by molding a mixture of the powdered compound moistened with an inert liquid. Desirably, each tablet may contain, for example, from about 0.1 mg to about 1.0 g of the active ingredient and each cachet or capsule contains from about 0.1 mg to about 500 mg of the active ingredient.

The GPR119 agonist alone or in combination with the drug (or combination of drugs) that reduces plasma glucose levels to below 70 mg/dL (or 60 mg/dL, 55 mg/dL or 50 mg/dL in particular embodiments) may be administered, by a route and in an amount commonly used, and when in combination contemporaneously or sequentially with the other. When the compounds are used contemporaneously, a combination pharmaceutical composition containing both GPR119 agonist and/or the drug (or combination of drugs) that reduces plasma glucose levels to below 70 mg/dL (or 60 mg/dL, 55 mg/dL or 50 mg/dL in particular embodiments) forms one embodiment hereof. In such an embodiment, the GPR119 agonist and/or drug (or combination of drugs) that reduces plasma glucose levels to below 70 mg/dL (or 60 mg/dL, 55 mg/dL or 50 mg/dL in particular embodiments) are present in a single dosage form. In specific embodiments thereof, the GPR119 agonist and/or drug (or combination of drugs) that reduces plasma glucose levels to below 70 mg/dL (or 60 mg/dL, 55 mg/dL or 50 mg/dL in particular embodiments) are in a bilayer tablet.

Alternatively, the GPR119 agonist and/or drug (or combination of drugs) that reduces plasma glucose levels to below 70 mg/dL (or 60 mg/dL, 55 mg/dL or 50 mg/dL in particular embodiments) are present each in a separate dosage form for simultaneous or sequential administration. In specific embodiments, the period of time between administration of the GPR119 agonist and/or the drug (or combination of drugs) that reduces plasma glucose levels to below 70 mg/dL (or 60 mg/dL, 55 mg/dL or 50 mg/dL in particular embodiments) is in the range from 0 minutes to 12 hours. The administration, when contemporaneously or sequentially, may be once, twice, three times or four times daily, per compound or combination.

The weight ratio of the GPR119 agonist and/or drug (or combination of drugs) that reduces plasma glucose levels to below 70 mg/dL (or 60 mg/dL, 55 mg/dL or 50 mg/dL in particular embodiments) may be varied within wide limits and depends upon the effective dose of each active ingredient. Generally, a therapeutically effective dose of each will be used. Thus, for example, when a GPR119 agonist is combined with a drug (or combination of drugs) that reduces plasma glucose levels to below 70 mg/dL (or 60 mg/dL, 55 mg/dL or 50 mg/dL in particular embodiments), the weight ratio of the GPR119 agonist to the drug (or combination of drugs) that reduces plasma glucose levels to below 70 mg/dL (or 60 mg/dL, 55 mg/dL or 50 mg/dL in particular embodiments) will generally range from about 1000:1 to about 1:1000, preferably about 200:1 to about 1:200.

A pharmaceutical composition which is present as a separate or multiple dosage form, preferably as a kit of parts, is useful in combination therapy to flexibly suit the individual therapeutic needs of the patient.

In a specific embodiment, a kit of parts comprises:
(a) a first containment containing a dosage form comprising the GPR119 agonist and at least one pharmaceutically acceptable carrier, and
(b) a second containment containing a dosage form comprising the drug (or combination of drugs) that reduces plasma glucose levels to below 70 mg/dL (or 60 mg/dL, 55 mg/dL or 50 mg/dL in particular embodiments) and at least one pharmaceutically acceptable carrier.

In a specific embodiment, a kit of parts comprises:
(c) a first containment containing a dosage form comprising the GPR119 agonist and at least one pharmaceutically acceptable carrier, and
(d) a second containment containing a dosage form comprising the insulin, insulin analog, or insulin secretagogue and at least one pharmaceutically acceptable carrier.
   In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In particular embodiments, the insulin secretagogue is a sulfonylurea compound. In particular embodiments, the insulin secretagogue is carbutamide, tolbutamide, tolazamide, chlorpropamide, acetohexamide, glyburide (also known as glibenclamide), glipizide, gliclazide, glibornuride, glipquidone, glisoxepide, glyclopyramide, or glimepiride. In specific embodiments, the present invention relates to a combination of GPR119 agonist, a non-sulfonylurea secretagogue and a pharmaceutically acceptable carrier. In specific embodiments, the insulin secretagogue is selected from Repaglinide or Nateglinide. In specific embodiments, the drug (or combination of drugs) that reduces plasma glucose levels to below 70 mg/dL (or 60 mg/dL, 55 mg/dL or 50 mg/dL in particular embodiments) is IDegLira.

A further aspect of the present invention is a manufacture comprising the pharmaceutical composition being present as separate dosage forms according to the present invention and a label or package insert comprising instructions that the separate dosage forms are to be administered contemporaneously or sequentially.

A yet further aspect of the present invention is a manufacture comprising a medicament which comprises a GPR119 agonist according to the present invention and a label or package insert which comprises instructions that the medicament may or is to be administered contemporaneously or sequentially with a medicament comprising a drug (or combination of drugs) that reduces plasma glucose levels to below 70 mg/dL (or 60 mg/dL, 55 mg/dL or 50 mg/dL in particular embodiments) according to the present invention. In particular embodiments, the drug (or combination of drugs) that reduces plasma glucose levels to below 70 mg/dL (or 60 mg/dL, 55 mg/dL or 50 mg/dL in particular embodiments) is an insulin, insulin analog, or insulin secretagogue. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In particular embodiments, the insulin secretagogue is a sulfonylurea compound. In particular embodiments, the insulin secretagogue is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In specific embodiments, the present invention relates to a combination of GPR119 agonist, a non-sulfonylurea secretagogue and a pharmaceutically acceptable carrier. In specific embodiments, the insulin secretagogue is selected from Repaglinide or Nateglinide. In specific embodiments, the drug (or combination of drugs) that reduces plasma glucose levels to below 70 mg/dL (or 60 mg/dL, 55 mg/dL or 50 mg/dL in particular embodiments) is IDegLira.

Another further aspect of the present invention is a manufacture comprising a medicament which comprises a drug (or combination of drugs) that reduces plasma glucose levels to below 70 mg/dL (or 60 mg/dL, 55 mg/dL or 50 mg/dL in particular embodiments) according to the present invention and a label or package insert which comprises instructions that the medicament may or is to be administered contemporaneously or sequentially with a medicament comprising a GPR119 agonist according to the present invention. In particular embodiments, the drug (or combination of drugs) that reduces plasma glucose levels to below 70 mg/dL (or 60 mg/dL, 55 mg/dL or 50 mg/dL in particular embodiments) is an insulin, insulin analog, or insulin secretagogue. In particular embodiments, the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218. In particular embodiments, the insulin secretagogue is a sulfonylurea compound. In particular embodiments, the insulin secretagogue is carbutamide, tolbutamide, tolazamide, chlorpropamide, acetohexamide, glyburide (also known as glibenclamide), glipizide, gliclazide, glibomuride, glipquidone, glisoxepide, glyclopyramide or glimepiride. In specific embodiments, the present invention relates to a combination of GPR119 agonist, a non-sulfonylurea secretagogue and a pharmaceutically acceptable carrier. In specific embodiments, the insulin secretagogue is selected from Repaglinide or Nateglinide. In specific embodiments, the drug (or combination of drugs) that reduces plasma glucose levels to below 70 mg/dL (or 60 mg/dL, 55 mg/dL or 50 mg/dL in particular embodiments) is IDegLira.

Synthesis of the compounds and development and manufacture of pharmaceutical compositions comprising same is well understood in the art.

The following examples are illustrative and provided so that the invention might be more fully understood. They should not be construed as limiting the invention in any way. The following abbreviations may be used in the Examples, Drawings, or the foregoing description: B.I.D. (bid or BID) is twice (or 2 times) a day; C57BL-Pan is C57BL mice pancreas; cmpd is compound; D₂O is deuterium oxide or heavy water; EGTA is ethyleneglycol-bis(2-aminoethylether)-N,N,N′,N′-tetraacetic acid; FACS is fluorescence activated cell sorting; FL1-H is fluorescence yellow fluorescent protein; FSC—H is forward scatter height value; GCG (or GcG) is glucagon; GPR-1R is glucagon like peptide 1 receptor; h is hours; i.p. is intraperitoneal; ISH is in situ hybridization; i.v. is intravenous; min is minutes; mpk is mg/kg; PBS is phosphate-buffered saline; PK is pharmacokinetic properties; p.o. is oral (by mouth); QD is once (or 1 time) a day; RPM is revolutions per minute; RT-PCT is reverse transcriptase-polymerase chain reaction; WT is wild-type; YFP is yellow fluorescent protein; and YFP-Pan is yellow fluorescent protein transgenic mice pancreas.

### Example 1

Role of GPR119 in Glucagon Release—Studies with GCG-YFP TG Mice

Methods

Islets were isolated from age-matched GcG-YFP transgenic and wild type (C57/b) mice; see Reimann et al., 2008 Cell Metab 8532-539. GCG-YFP Tg mice express YFP protein under the control of the preproglucagon promoter, 150-200 islets were picked and transferred to a 1.5 mL EPPENDORF tube. Islets were incubated in 0.5 ml diluted IX trypsin dispersing medium at 37° C. for 10 minutes. Islet cells were dispersed by passing through a fine-tip pipette (flat gel loading tips, 0.17 mm), ˜50 pipettings. Cells were centrifuged for 5 minutes at 5000 rpm and re-suspended in ˜0.5 ml prewarmed dispersion solution with 1 mM EGTA ( 1/500 vol 100 mM EGTA, pH 8.0). If needed, cells were further incubated for an additional 10 minutes at 37° C. until small dense clusters of islets were no longer visible. Where big clumps were present, more pipettings were applied. The solution was centrifuged at 5000 RPM for 5 minutes, and the pellet was dispersed in 0.5 ml PBS in preparation for the FACS study; see Reimann et al. supra. Sorted cells were lysed for RNA purification and Tagman gene expression analysis; see Reimann et al., supra.

Results

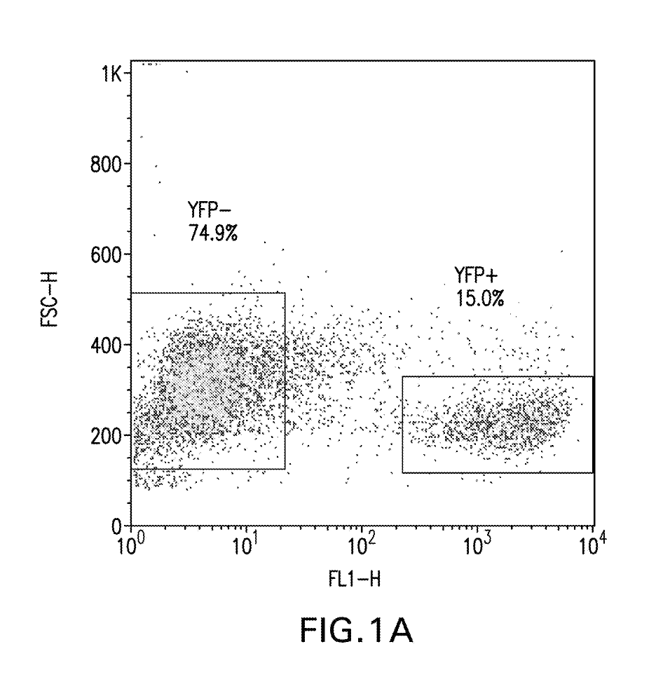

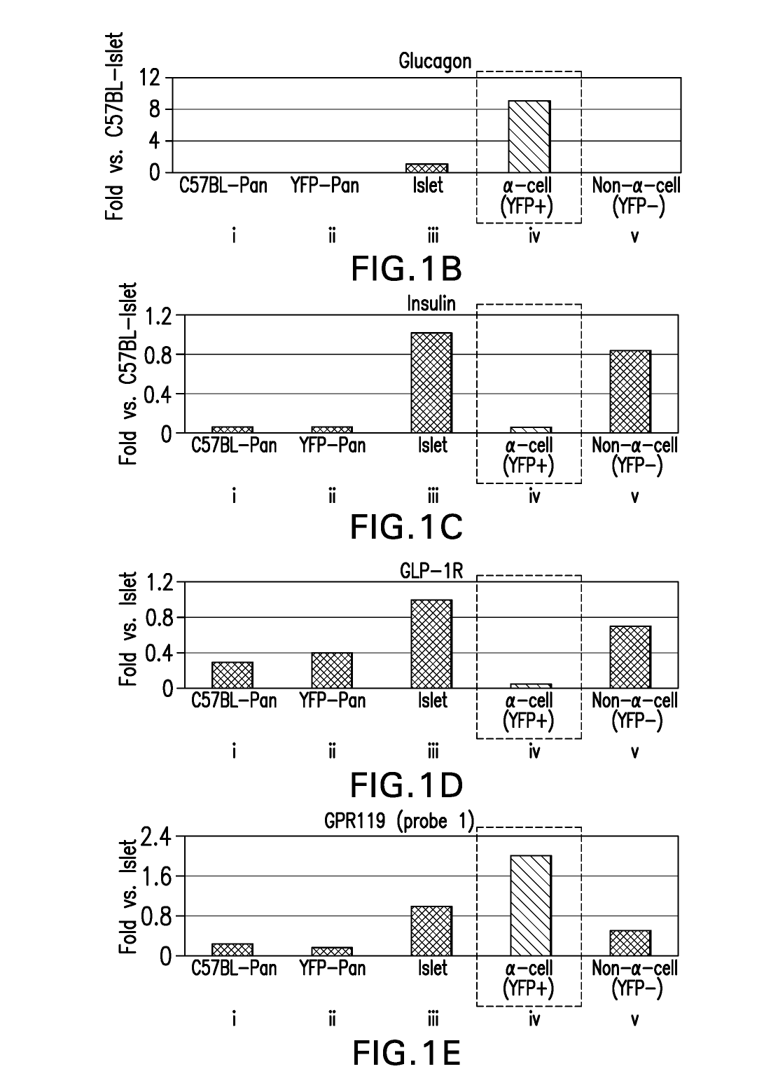

FIGS. 1A-E illustrate how GPR119 is highly expressed in pancreatic α-cells. FIG. 1A is a representative FACS plot showing two subsets (YFP+, 15% and YFP−, 74.9%) of islet cells separated from purified islets of GCG-YFP Tg mice. GCG-YFP Tg mice express YFP protein under the control of the preproglucagon promoter. Therefore. YFP+ cells are glucagon⁺ α-cells. FIGS. 1B-1E illustrate relative mRNA levels of the indicated genes, measured by TAQMAN RT-PCR, in total pancreata from (i) wildtype (C57BL/6) mice, (ii) GCG-YFP Tg mice, and in (iii) purified islets, (iv) FACS sorted YFP+ cells, and (v) FACS-sorted YFP− cells from GCG-YFP Tg mice, respectively.

YFP+ cells are notable for the abundance of glucagon mRNA, but lack of insulin and GLP-1R expression. Surprisingly. GPR119 expression is enriched in YFP⁺ glucagon⁺ α-pancreatic cells, as compared with YFP⁻ non-α-pancreatic cells. These results have been repeated in 3 different experiments.

### Example 2

### GPR119 Expression in In Situ Hybridization (ISH) Studies

Formalin fixed paraffin embedded (FFPE) mouse, rhesus monkey and human pancreas tissue sections were analyzed. Human pancreata were received from the network for Pancreatic Organ Donors with Diabetes (nPOD). Only tissues with RNA Integrity scores (RIN)>7 were studied. Three mouse pancreata, three monkey pancreata and seven donors were chosen for the study, N=2 for non-diabetics, N=4 for Type 1 diabetic donors, and N=1 for Type 2 diabetic donors. Duplex in situ hybridization was applied to analyze by immunohistochemistry (IHC) the co-expression of GPR119 mRNA with glucagon, insulin, or somatostatin in pancreatic islets corresponding to the α, β, and δ cells of the pancreas respectively. This was carried out using the RNAscope® 2.0 Assay 2-plex kit and probes from Advanced Cellular Diagnostics (ACD) (Wang F, Flanagan J, Su N, Wang L-C, Bui S. Nielson A, Wu X, Vo H-T, Ma X-J and Luo Y. RNAscope®: A Novel In Situ RNA Analysis Platform for Formalin-Fixed Paraffin-Embedded Tissues. J. Mol. Diagnostics, 2012, 14:22-29). Detailed protocols for sample preparation and the duplex ISH can be found in the RNAscope® Sample Preparation and Pretreatment Guide for FFPE Tissue, PART 1 and the RNAscope® 2-Plex Detection Kit (Chromogenic) User Manual. A positive control probe, commercially available peptidylprolyl isomerase B (PPIB), was used to qualify the mRNA integrity of each tissue sample. All sections were counterstained with hematoxylin.

Results

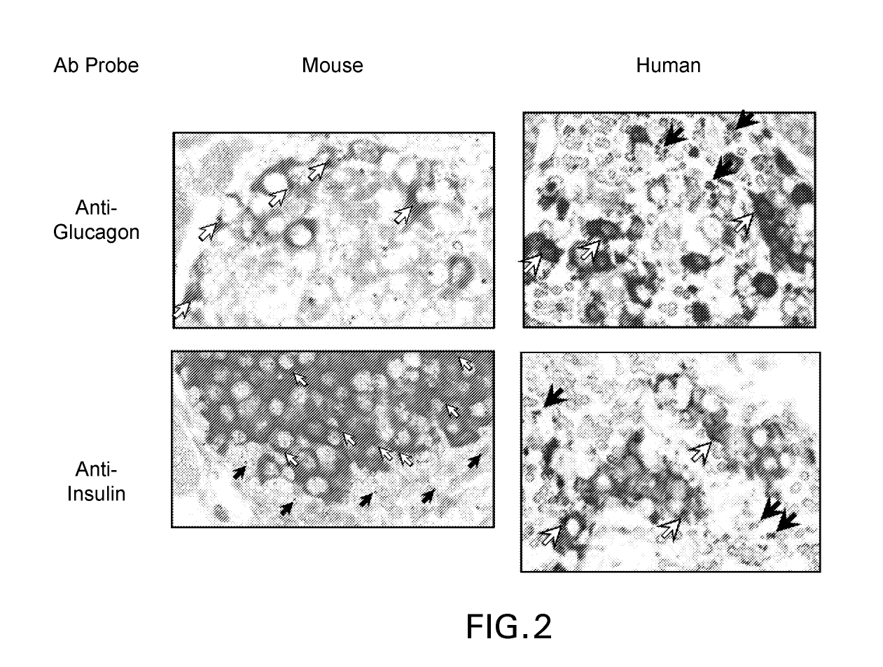

GPR119 mRNA is co-expressed in α- and β-cells from mouse and human pancreata; see FIG. 2. Pancreata from wild-type mice and normal human subjects were immunostained for glucagon (top panels, dark staining in mouse on left and human on right), and insulin (lower panels, dark staining in mouse on left and human on right). The pancreata were additionally co-currently subjected to in situ hybridization of GPR119 mRNA (lighter staining). As noted, GPR119 mRNA is co-expressed in glucagon and insulin positive cells (white arrows outlined in black).

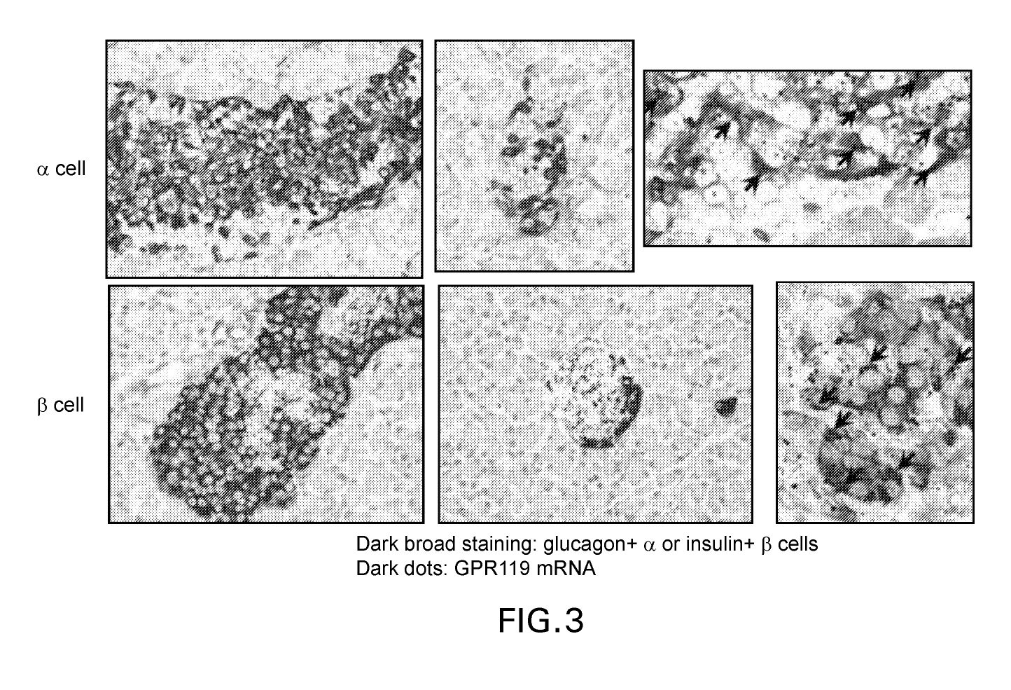

FIG. 3 illustrates GPR119 expression in both α and β cells of rhesus monkey pancreatic islet cells. Monkey pancreata were immunostained for glucagon (top panels, dark broad staining) or insulin (bottom panels, dark broad staining), and were co-currently subjected to in situ hybridization of GPR119 mRNA (dark dots). GPR119 mRNA was found to be co-expressed in glucagon and insulin positive cells (black arrows).

### Example 3

### Effect of GPR119 Agonists on Rat Pancreatic Perfusion

Methods

Male Wistar Han rats were purchased from Charles River Laboratories Inc. at 6 weeks of age and housed 2 per cage for two weeks with food and water ad libitum. In Situ Pancreatic perfusions were performed when the rats were eight weeks old. All procedures were performed in accordance with relevant guidelines and regulations. Food was removed ˜4-5 hours prior to surgery. For each surgery, the rat was fully sedated with 100 mg/kg intraperitoneal (i.p.) Na-pentobarbital (NEMBUTAL) anesthesia. The depth of anesthesia was determined by a “paw pinch” test. Surgery was not initiated until the rat was fully sedated. If necessary an additional 50 mg/kg i.p. dose (50% of original) of NEMBUTAL was given. The peritoneal cavity was opened and coeliac artery was ligated dorsally. A 27G cannula was inserted into coeliac artery for perfusant afflux. The left gastric artery was ligated at the esophagus-stomach junction. The hepatic portal vein was ligated dorsally and cannulated ventrally (25G cannula; towards liver) for perfusant efflux. Immediately following the surgery, the rat was placed into the perfusion chamber and perfused at 3 ml/minute with modified KREBS-RINGER buffers [see. Geisler et al. 2012 Reproductive biology and endocrinology 10:110]. The buffers and perfusion chamber were kept warm at 37° C. and saturated with an O²/CO² gases mixture. The buffers were 2 mM or 16 mM glucose and contained either vehicle (0.1%/DMSO) or Compound A (see Table 1) or 30 mM L-Arginine. The perfusant was collected by automated fraction collector at one sample per minute rate with about 90% rate of buffer recovery. Samples were frozen at −70° C. and later analyzed for total insulin and glucagon content.

For the buffers: First the following mixture was prepared in water containing 10% NaCl (1.19M), 5% KCL (94 mM), 10% NaHCO₂ (250 mM), 5% CaCl₂-2H₂O (50 mM), 5% MgSO₄-7H₂O (24 mM), and 5% KH₂PO₄ (24 mM). The buffer was then oxygenated for 20 minutes, and 3% dextran and 0.2% BSA (insulin free) were added. Glucose was added from 1M stock solution as needed.

Results

A GPR119 agonist enhances islet glucagon release in a rat pancreatic perfusion model.

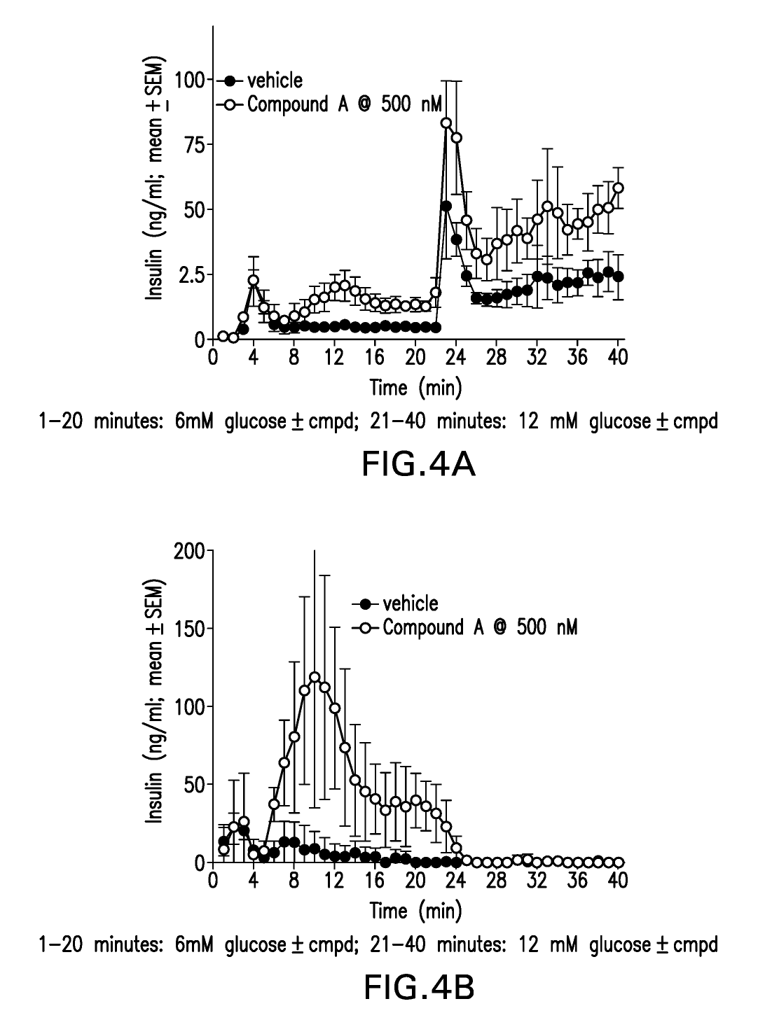

FIGS. 4A-B illustrate a pancreatic perfusion assay in Wistar Han rats, and insulin (left panel) and glucagon (right panel) release measurements. As shown (lines with open circles), a basal level of insulin and a fair amount of glucagon were secreted from pancreas when perfused with low glucose (6 mM glucose). Once the pancreas was infused with high glucose (12 mM glucose), pancreas insulin secretion was dramatically induced, accompanied with repressed glucagon release. Application of GPR119 agonist (500 nM Compound A, lines with solid circles) enhanced both basal and glucose-stimulated pancreas insulin secretion (left panel). In addition, compound elevated basal glucagon release, but did not affect glucose-repressed glucagon secretion from pancreas (right panel). Hence, in addition to the enhancement on insulin secretion, activation of GPR119 promotes basal glucagon release from pancreatic islets. This study demonstrates the ability of GPR119 agonists to regulate both glucagon and insulin secretion in a glucose dependent manner (stimulating secretion of insulin or glucagon depending on the glucose levels of the environment), which may allow greater control over blood glucose levels within a physiological range.

### Example 4

### Hyperinsulinemic-Hypoglycemia Clamp Study

Methods

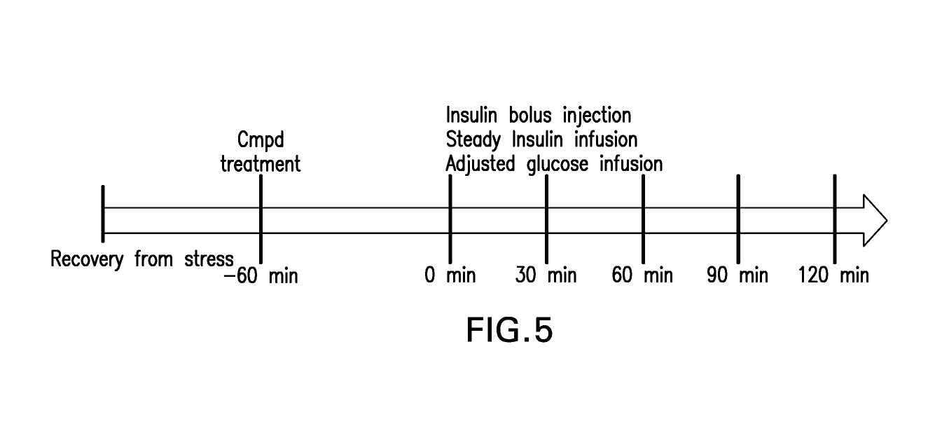

A hyperinsulinemic-hypoglycemia clamp study was performed in male Wistar Han rats as shown in FIG. 5 and described below to explore the potential roles of GPR119 agonists in regulating glucagon secretion at the hypoglycemic status. Three groups were studied, those treated with: (1) vehicle, (2) GPR119 agonist, Compound B, C, F, H, I, M and V (see Table 1 and Table 2), at 10, 30, 100, 100, 100, 30 and 30 mpk, respectively, or (3) GLIBENCLAMIDE, a sulfonylurea insulin secretagogue known to cause hypoglycemia, at 5 mpk. Blood samples (100-150 μl) were collected for insulin, C-peptide, glucagon, and epinephrine/norepinephrine measurements. Terminal bleeding was analyzed for PK measurement.

Results

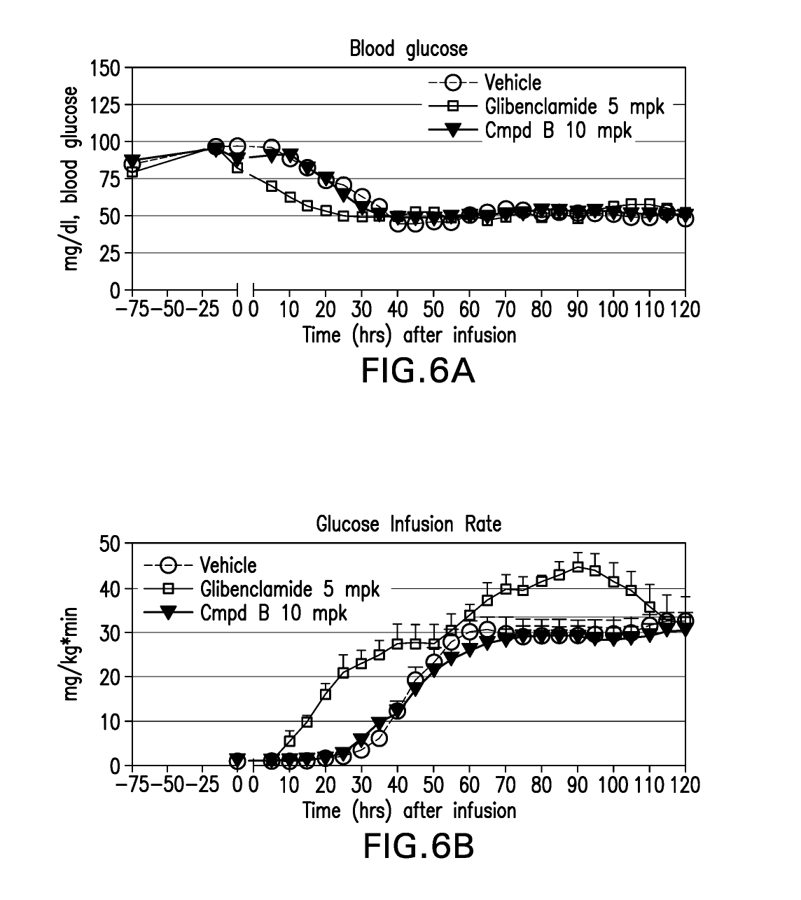

FIGS. 5, 6A-B, 7A-D and 8A-L illustrate the hyperinsulinemic-hypoglycemia clamp study of EXAMPLE 4. FIG. 5 is a scheme and time-course depiction of the hyperinsulinemic-hypoglycemia clamp study performed in rats. Vertical bars indicate the time points when the rats were infused with compound, or insulin/glucose, or blood specimens were collected for measurements of insulin, C-peptide, glucagon, and epinephrine/norepinephrine. After recovered from surgery stress, rats were pre-treated with vehicle, GLIBENCLAMIDE (5 mpk), or GPR119 agonist Compound B (10 mpk) 60 minutes prior to initiation of the clamp. From time zero, rats received an initial insulin bolus injection followed by simultaneous infusion of insulin at a steady rate and glucose at an adjusted rate. Rat plasma glucose levels (FIG. 6A) were monitored periodically, and the glucose infusion rate (FIG. 6B) was adjusted so that the blood glucose level would be maintained at 50 mg/dl. Unlike GLIBENCLAMIDE which rapidly decreases blood glucose level and requires much faster glucose infusion rate, the changes of systemic blood glucose level and glucose infusion rate were comparable and slower in rats received with vehicle or GPR119 agonist Compound B, suggesting that agonism of GPR119 may cause less risk of hypoglycemia.

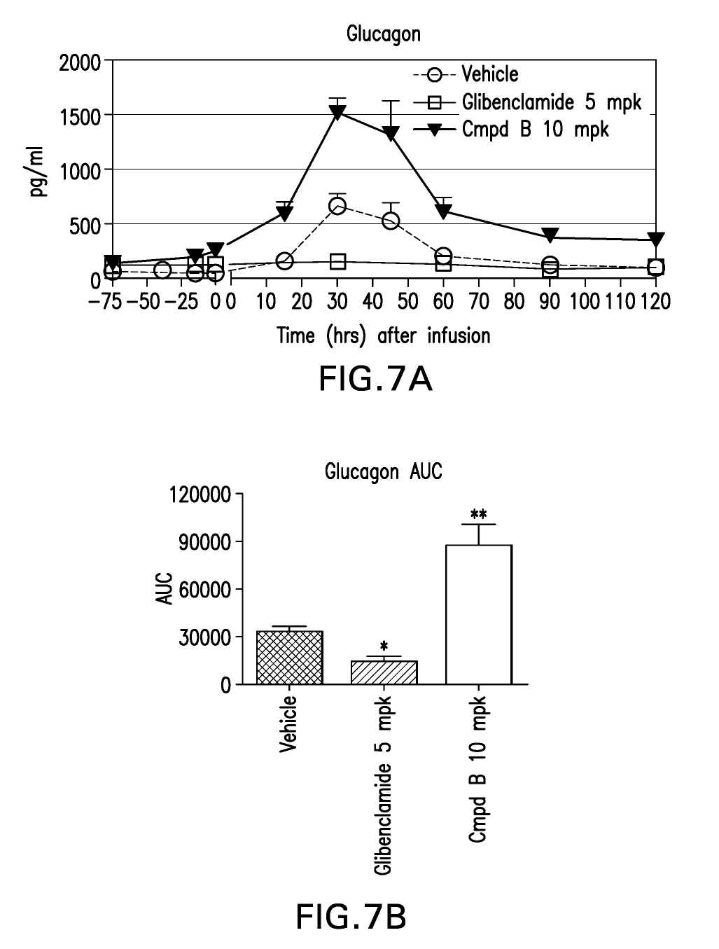

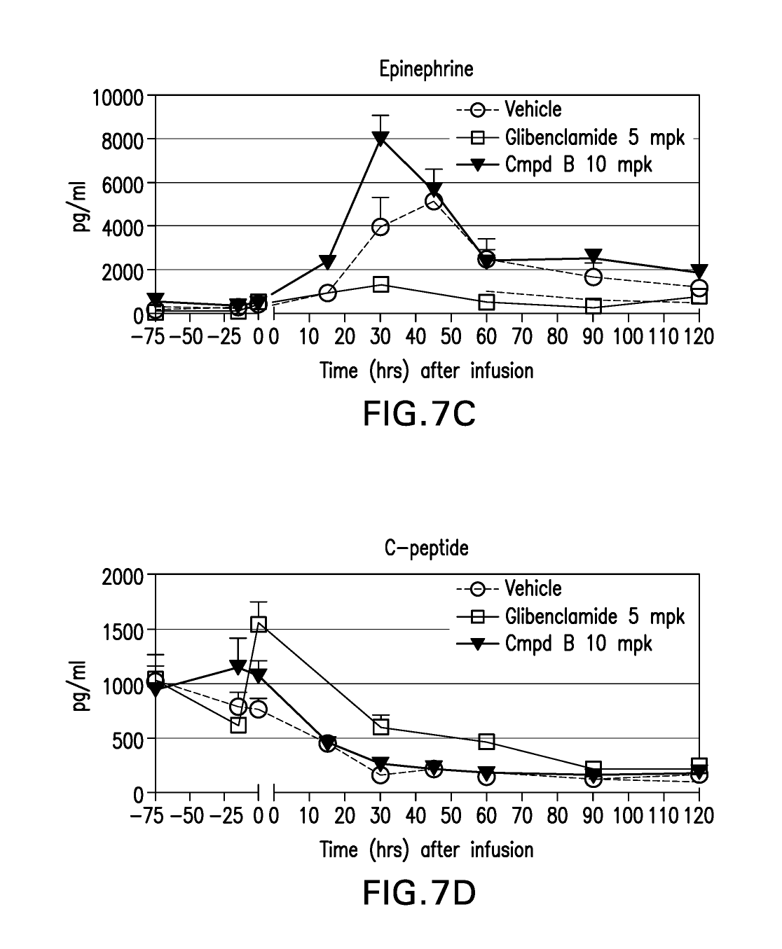

GPR119 agonist, furthermore, significantly enhanced glucagon release at the hypoglycemic status; whereas GLIBENCLAMIDE suppressed glucagon secretion. FIGS. 7A-D illustrate measurements of blood glucagon (FIGS. 7A-B), epinephrine (FIG. 7C), and c-peptide (FIG. 7D) levels in rats received from the clamp study. A transient increase of glucagon release was observed between 15-60 minutes in clamped rats when their blood glucose levels were dropping to 50 mg/dL. GPR119 agonist Compound B significantly elevated glucagon secretion at this hypoglycemic status (FIGS. 7A-B). GLIBENCLAMIDE blunted this response (FIGS. 7A-B). In contrast, rat systemic epinephrine levels was also elevated at this hypoglycemic status (FIG. 7C). GLIBENCLAMIDE still inhibited this elevation; however, GPR119 agonist Compound B only slightly altered epinephrine release (FIG. 7C). As expected, GLIBENCLAMIDE promotes c-peptide release. The impact of GPR119 agonist Compound B on c-peptide release was notably indistinguishable from vehicle control (FIG. 7D).

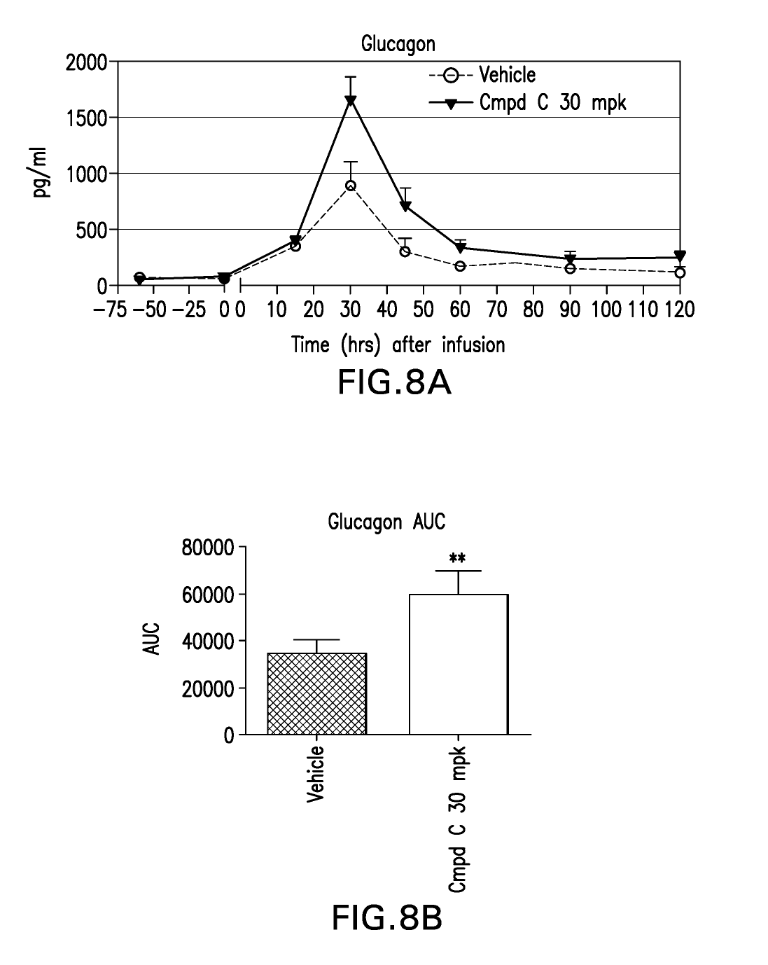

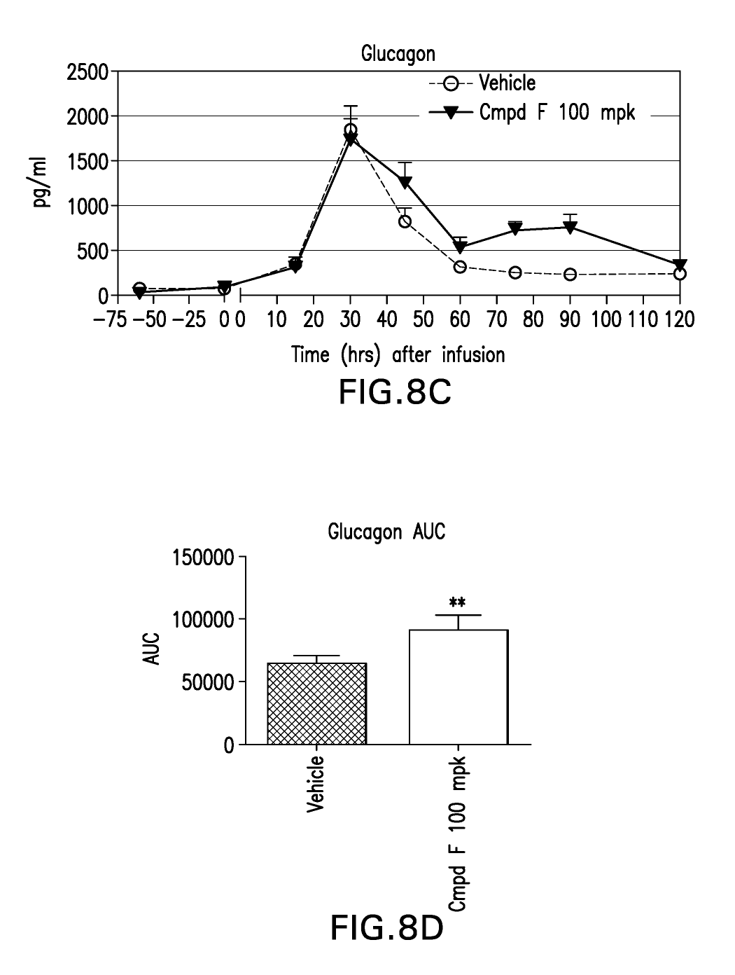

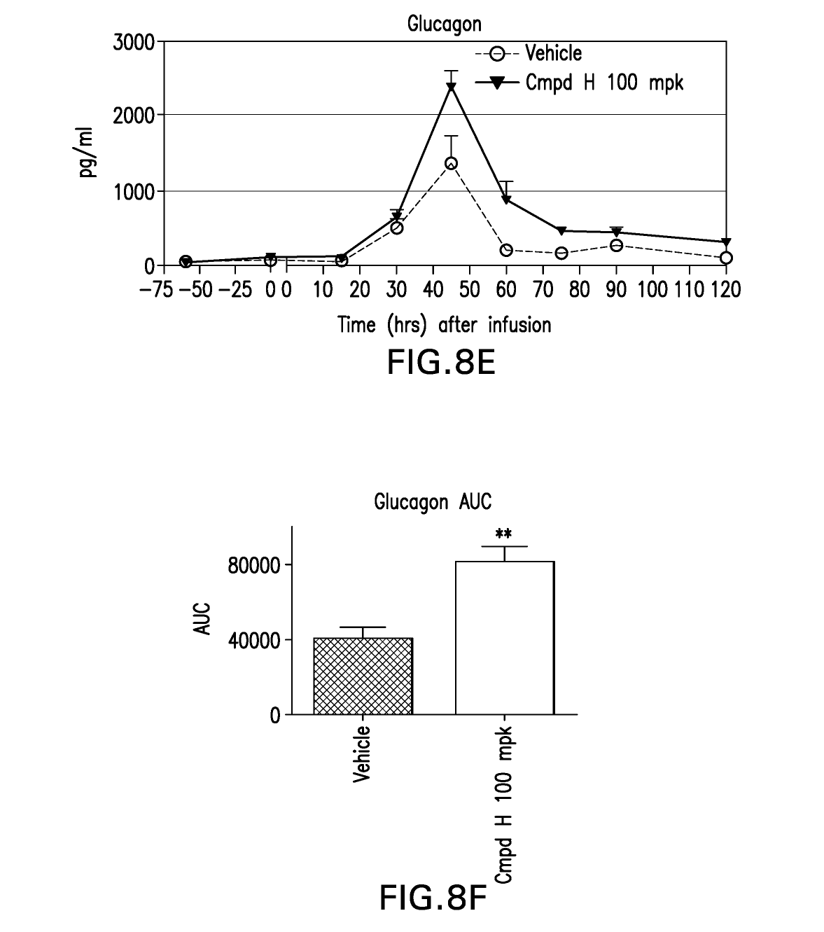

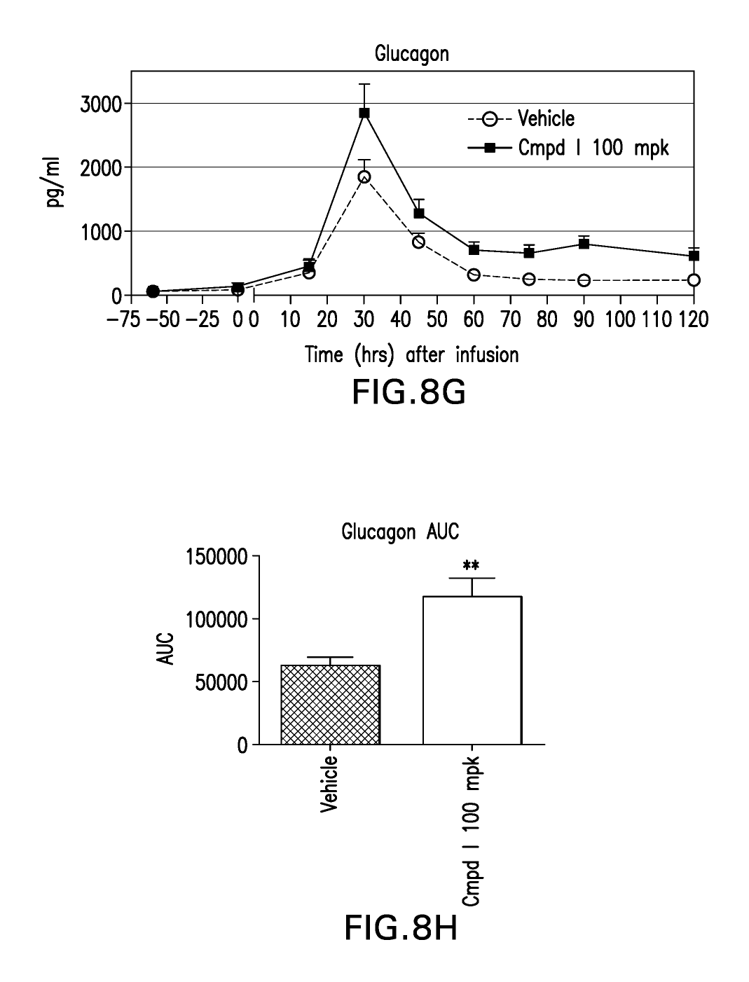

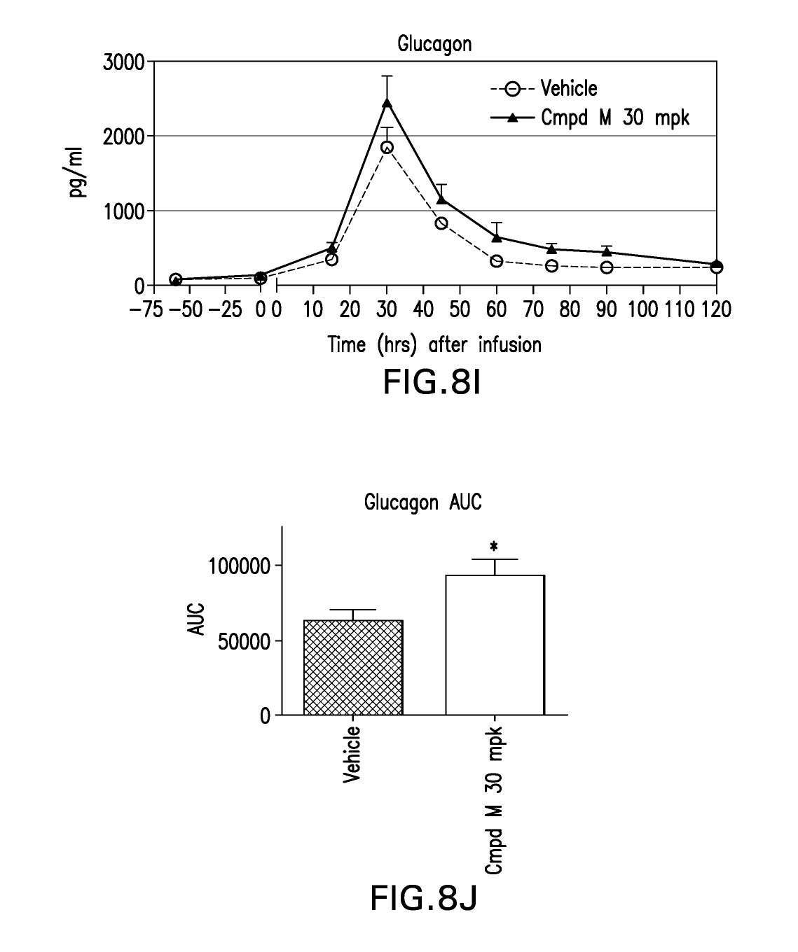

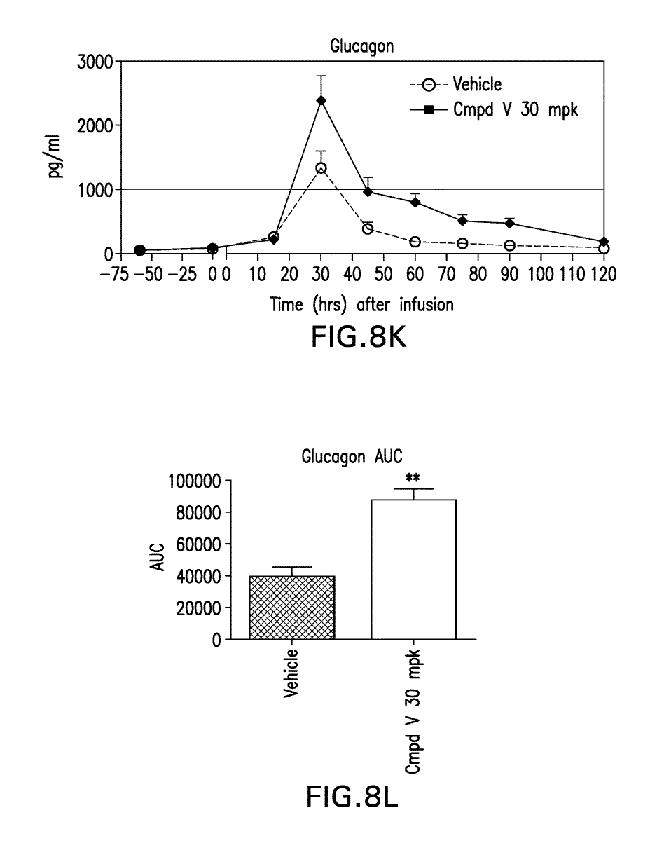

Similarly, at the same hypoglycemic status (50 mg/dL), GPR119 agonist Compounds C, F, H, I, M and V significantly elevated glucagon secretion (FIGS. 8A-L).

These results indicate that GPR119 agonists specifically enhance islet glucagon release in the hypoglycemic state. Thus, GPR119 agonists are particularly useful to prevent or treat hypoglycemia in diabetic patients. Supplemented to insulin, insulin analogs, or insulin secretagogues, all of which carry a risk of hypoglycemia, the GPR119 agonists may prevent the onset of hypoglycemia through the stimulation of glucagon release in the context of decreasing glucose levels.

Certain embodiments of the invention have been described in detail; however, numerous other embodiments are contemplated as falling within the invention. Thus, the claims are not limited to the specific embodiments described herein. All patents, patent applications and publications that are cited herein are hereby incorporated by reference in their entirety.

1. A method of preventing hypoglycemia which comprises administering a pharmaceutical composition comprising a GPR119 agonist compound and a pharmaceutically acceptable carrier to a subject at risk for hypoglycemia.

2. A method of preventing hypoglycemia which comprises administering a pharmaceutical composition comprising a GPR119 agonist compound and a pharmaceutically acceptable carrier to a subject with a history of hypoglycemia.

3. A method of treating hypoglycemia which comprises administering a pharmaceutical composition comprising a GPR119 agonist compound and a pharmaceutically acceptable carrier to a subject having a plasma glucose level less than 70 mg/dL.

4. The method of claim 1 wherein the subject is being treated with insulin, an insulin analog or an insulin secretagogue.

5. The method of claim 4 wherein the insulin or insulin analog is selected from human insulin, NPH insulin, insulin lispro, insulin aspart, insulin glulisine, insulin detemir, insulin glargine, insulin degludec, insulin peglispro, insulin known as NN1436 (LA1287), or insulin known as NN1218.

6. The method of claim 1 wherein the subject is being treated with an insulin secretagogue which is a sulfonylurea or non-sulfonylurea compound.

7. The method of claim 6 wherein the sulfonylurea or non-sulfonylurea compound is carbutamide, tolbutamide, tolazamide, chlorpropamide, acetohexamide, glyburide, glipizide, gliclazide, glibornuride, glipquidone, glisoxepide, glyclopyramide, glimepiride, Repaglinide or Nateglinide.

8. The method of claim 1 wherein the subject has been diagnosed with Type 1 Diabetes.

9. The method of claim 8 where the subject is being treated with insulin, an insulin analog or an insulin secretagogue.

10. The method of claim 1 wherein the subject has been diagnosed with Type 2 Diabetes.

11. The method of claim 10 where the subject is being treated with insulin, an insulin analog or an insulin secretagogue.

12. The method of claim 1 wherein the GPR119 agonist is: 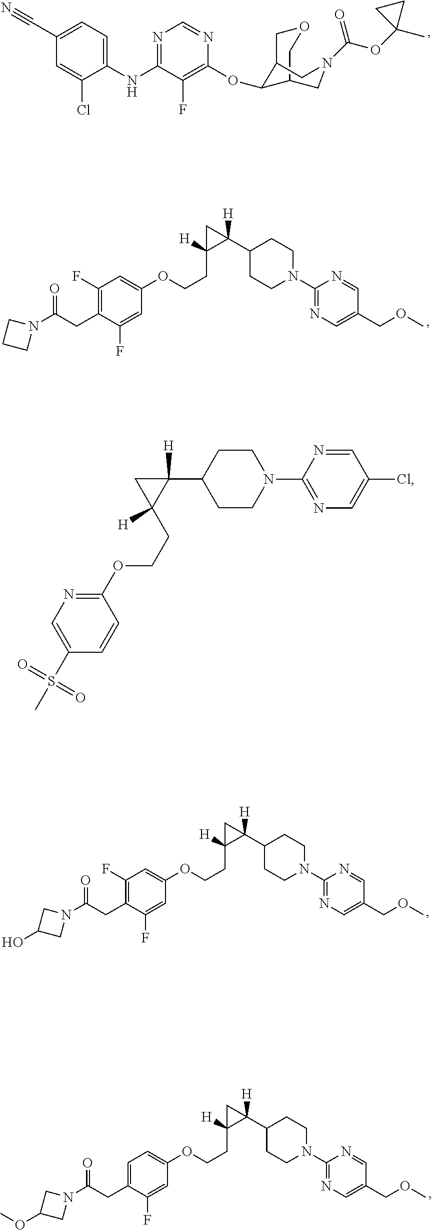 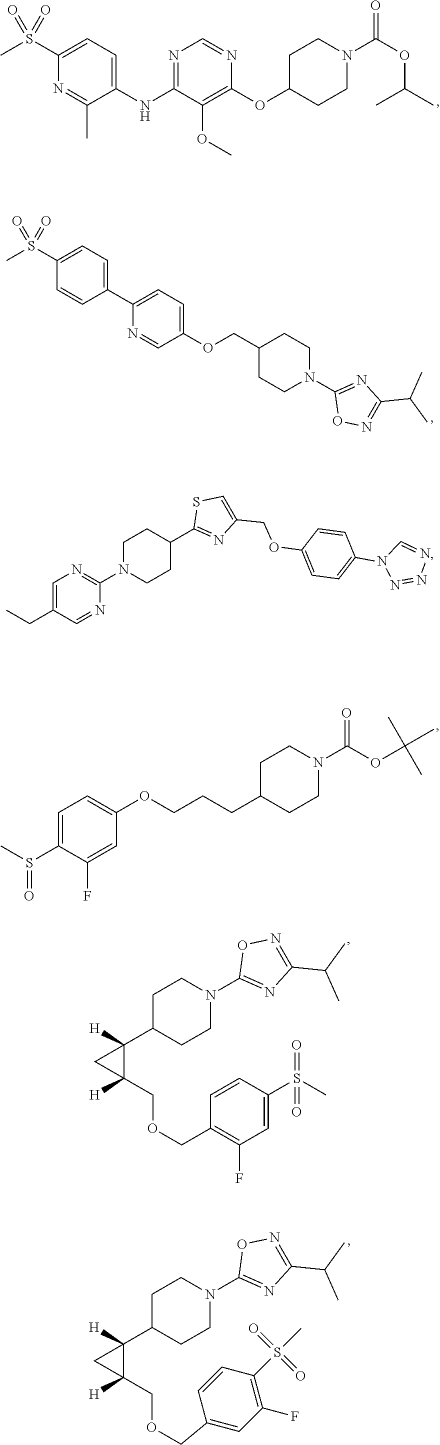 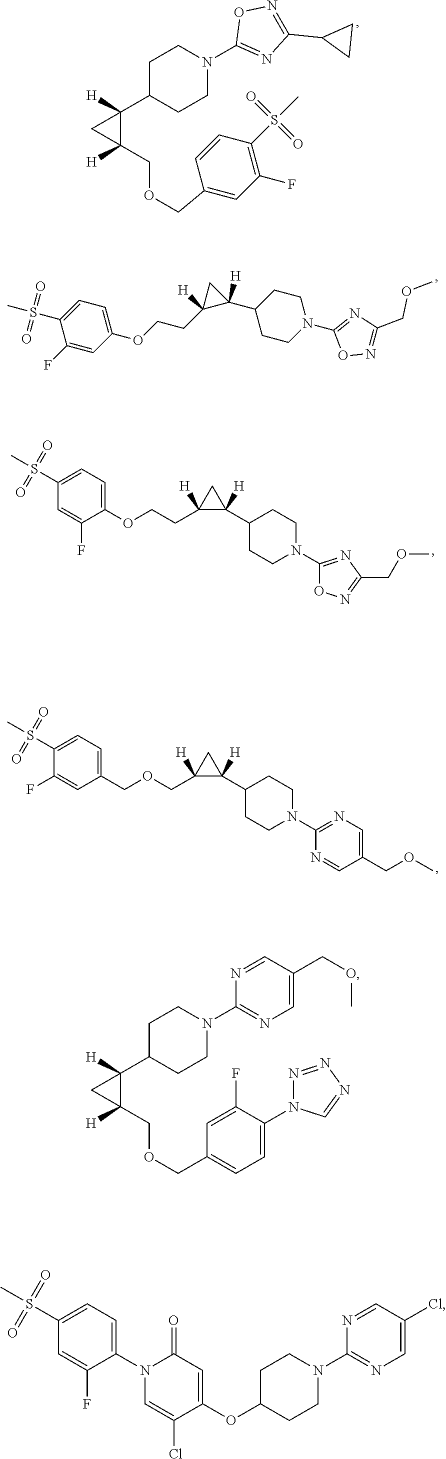 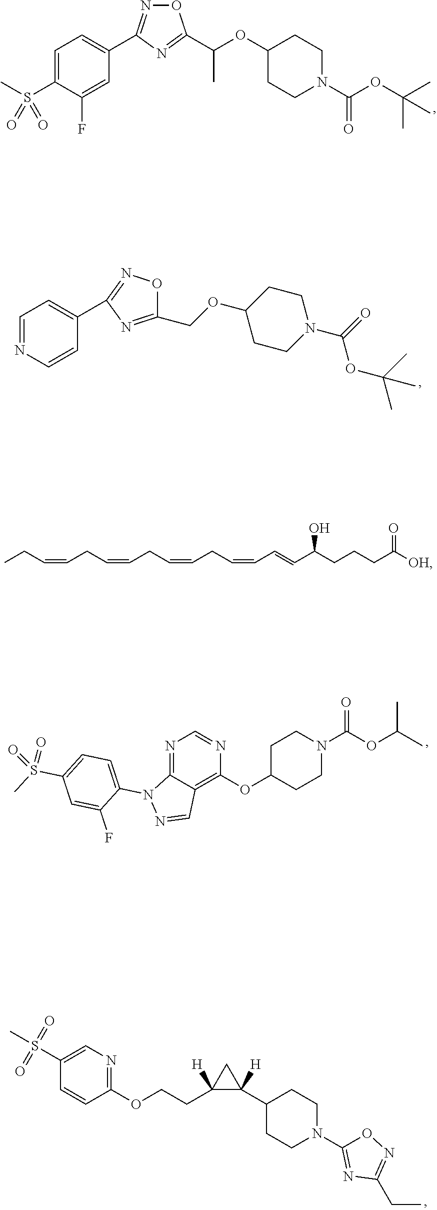
or a pharmaceutically acceptable salt thereof.

13. The method of claim 12 where the subject is being treated with insulin, an insulin analog or an insulin secretagogue.

14. An article of manufacture that comprises a container holding:
(a) a pharmaceutical composition comprising a GPR119 agonist and a pharmaceutically acceptable carrier; and
(b) printed instructions for preventing hypoglycemia in a subject at risk for hypoglycemia by using the pharmaceutical composition.

15. The article of manufacture of claim 14 wherein the subject is treated with insulin, an insulin analog or an insulin secretagogue.

16. An article of manufacture that comprises a container holding:
(a) a pharmaceutical composition comprising a GPR119 agonist and a pharmaceutically acceptable carrier; and
(b) printed instructions for treating hypoglycemia in a subject diagnosed as having hypoglycemia by using the pharmaceutical composition.

17. The article of manufacture of claim 16 wherein the subject is treated with insulin, an insulin analog or an insulin secretagogue.

18-20. (canceled)

21. The method of claim 12 wherein the GPR1219 agonist is: 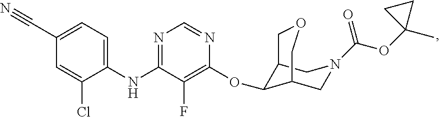 [SMILES: CC1(OC(=O)N2CC3COCC(C2)C3Oc2ncnc(Nc3ccc(C#N)cc3Cl)c2F)CC1]
or a pharmaceutically acceptable salt thereof.

22. The method of claim 12 wherein the GPR1219 agonist is: 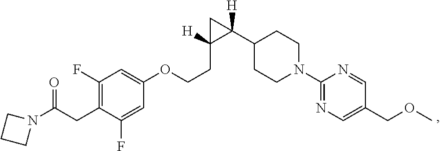 [SMILES: COCc1cnc(N2CCC([C@H]3C[C@H]3CCOc3cc(F)c(CC(=O)N4CCC4)c(F)c3)CC2)nc1]
or a pharmaceutically acceptable salt thereof.
